(19) United States
(12) Patent Application Publication
(10) Pub. No.: US 2017/0273932 A1
(43) Pub. Date: Sep. 28, 2017

(54) LYOPHILIZED COMPOSITIONS CONTAINING A METAP-2 INHIBITOR

(71) Applicant: Zafgen, Inc., Boston, MA (US)

(72) Inventors: James E. Vath, Lynnfield, MA (US); Michelle Howard-Sparks, Newton, MA (US); Nancy Harper, Taunton, MA (US); David Dresback, Stonington, CT (US); Thomas Crawford, Essex, CT (US); Gary Elliott, Portland, ME (US); Walter Lunsmann, Harvard, MA (US); Grant Wilson, San Diego, CA (US); Jose Casillas, Chula Vista, CA (US)

(21) Appl. No.: 15/505,308

(22) PCT Filed: Aug. 24, 2015
(86) PCT No.: PCT/US2015/046547

§ 371 Date: Feb. 21, 2017

Related U.S. Application Data
(60) Provisional application No. 62/040,877, filed on Aug. 22, 2014.

(51) Int. Cl. A61K 31/336; A61K 47/10; A61K 47/26; A61K 47/14; A61K 9/00; A61K 47/24; A61K 47/32; A61K 47/38; A61K 47/02; A61K 9/19; A61K 47/44

(52) U.S. Cl. CPC A61K 31/336; A61K 9/19; A61K 47/10; A61K 47/26; A61K 47/14; A61K 47/44; A61K 47/24; A61K 47/32; A61K 47/38; A61K 47/02; A61K 9/0095; A61K 9/0019

(57) ABSTRACT

This disclosure features compositions (e.g., re-constitutable solids, such as lyophilized cakes; or liquid compositions, such as solutions or suspensions, e.g., reconstituted lyophilized cakes) that are suitable for subcutaneous administration. The compositions include a crystalline form (e.g., Form A) of 6-O-(4-dimethylaminoethoxy)cinnamoyl fumagillol, free base and one or more pharmaceutically acceptable excipients and/or one or more pharmaceutically acceptable carriers (e.g., one or more bulking agents; one or more dispersing agents; one or more buffers; one or more suspending agents; water, e.g., water for injection (“WFI”)).

### CROSS REFERENCE TO RELATED APPLICATIONS

This application claims priority to U.S. provisional patent application Ser. No. 62/040,877, filed Aug. 22, 2014, which is hereby incorporated by reference in its entirety.

### BACKGROUND

6-O-(4-Dimethylaminoethoxy)cinnamoyl fumagillol is a METAP2 inhibitor and is useful in the treatment of, e.g., obesity. 6-O-(4-Dimethylaminoethoxy)cinnamoyl fumagillol is characterized by formula I:

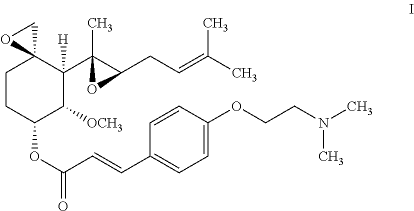 [SMILES: CO[C@@H]1[C@H](OC(=O)/C=C/c2ccc(OCCN(C)C)cc2)CC[C@]2(CO2)[C@H]1[C@@]1(C)O[C@@H]1CC=C(C)C]

Crystalline forms of the free base of 6-O-(4-Dimethylaminoethoxy)cinnamoyl fumagillol are described, e.g., in U.S. Pat. No. 8,349,891, U.S. Pat. No. 8,735,447, and WO 2012/064838, each of which is incorporated herein by reference in its entirety.

While these compounds have shown promise in the treatment of obesity, delivery of such compounds is challenging. For example, intravenous administration is not a viable delivery option in some obese patients due to difficulties associated with consistent venous access. Oral delivery is not a viable option as well due to the extremely low and variable bioavailability of these compounds following oral administration—both when delivered to the stomach or to the duodenum in a buffered solution.

There is a need therefore, for a subcutaneous delivery form that also provides for substantial bioavailability. Such a drug composition should be substantially stable. Importantly, such formulations, when delivered to patient, should also be deliverable through a smaller needle to avoid unnecessary pain at administration. Because in part no commercial suspension lyophile has been developed, the discovery challenge of a suspension lyophile that meets these requirements is high.

### SUMMARY

This disclosure features compositions such as lyophilized compositions; or suspensions, e.g., reconstituted lyophilized cakes, that are suitable for subcutaneous administration. Disclosed compositions include a crystalline form (e.g., Form A) of 6-O-(4-dimethylaminoethoxy)cinnamoyl fumagillol, free base (or alternatively, a crystalline form of a pharmaceutically acceptable salt and/or hydrate and/or/solvate of 6-O-(4-dimethylaminoethoxy)cinnamoyl fumagillol), and one or more pharmaceutically acceptable excipients and/or one or more pharmaceutically acceptable carriers (e.g., one or more bulking agents; one or more dispersing agents; one or more buffers; one or more suspending agents; water, e.g., water for injection (“WFI”)). Said crystalline form and the one or more pharmaceutically acceptable excipients and/or the one or more pharmaceutically acceptable carriers are sometimes referred to herein as “components” of the compositions. In some embodiments, the compositions can be in the form of a re-constitutable solid, e.g., a lyophilized cake (sometimes referred to herein as a “lyophile”). In other embodiments, the compositions can be liquid compositions, such as solutions or suspensions, e.g., a reconstituted lyophile (e.g., reconstituted with water, e.g., WFI; and optionally one or more pharmaceutically acceptable excipients).

Accordingly, in one aspect, this disclosure features suspension lyophile compositions suitable for subcutaneous administration to a patients upon reconstitution, which include (i) a crystalline form (e.g., Form A) of 6-O-(4-dimethylaminoethoxy)cinnamoyl fumagillol, free base; (ii) a bulking agent (which e.g., may promote manufacturing of a stable lyophile cake); and (iii) a dispersing agent (which e.g., may promote dispersion of crystalline form).

In another aspect, this disclosure features suspension lyophile compositions suitable for subcutaneous administration to a patient upon reconstitution, which include (i) a crystalline form (e.g., Form A) of 6-O-(4-dimethylaminoethoxy)cinnamoyl fumagillol, free base; (ii) about 78 to about 88 weight percent trehalose or trehalose dihydrate; and/or (iii) about 1 to 2 weight percent poloxamer 188.

In a further aspect, this disclosure features a pre-loaded syringe, which includes any one of the suspension lyophile compositions described herein. In some embodiments, the syringe can be a dual chamber syringe and one chamber of the syringe contains the lyophile suspension composition.

In one aspect, this disclosure features a reconstitution vial, which includes any one of the suspension lyophile compositions described herein.

In another aspect, this disclosure features ready to use reconstituted suspension compositions, which include any one of the suspension lyophile compositions described herein and a reconstitution vehicle. In some embodiments, the reconstitution vehicle can include water and non-ionic polymer, e.g., water and polysorbate 80.

In a further aspect, this disclosure features a unit dose vial or pre-loaded syringe for delivering about a 6 mg drug product dose to a patient, which includes any one of the suspension lyophile compositions described herein, wherein the composition has about 7 mg of the crystalline form (e.g., Form A) of the compound 6-O-(4-dimethylaminoethoxy)cinnamoyl fumagillol, free base.

In one aspect, this disclosure features a unit dose vial or pre-loaded syringe for delivering about a 4.5 mg drug product dose to a patient, which includes any one of the suspension lyophile compositions described herein, wherein the composition has about 5.6 mg of the crystalline form (e.g., Form A) of the compound 6-O-(4-dimethylaminoethoxy)cinnamoyl fumagillol, free base.

In another aspect, this disclosure features a unit dose vial or pre-loaded syringe for delivering about a 3.2 mg drug product dose to a patient, which includes any one of the suspension lyophile compositions described herein, wherein the composition has about 4 mg of the crystalline form (e.g., Form A) of the compound 6-O-(4-dimethylaminoethoxy)cinnamoyl fumagillol, free base.

In a further aspect, this disclosure features a unit dose vial or pre-loaded syringe for delivering about a 2.8 mg drug product dose to a patient, which includes any one of the suspension lyophile compositions described herein, wherein the composition has about 3.5 mg of the crystalline form (e.g., Form A) of the compound 6-O-(4-dimethylaminoethoxy)cinnamoyl fumagillol, free base.

In one aspect, this disclosure features a unit dose vial or pre-loaded syringe for delivering about a 2.4 mg drug product dose to a patient, which includes any one of the suspension lyophile compositions described herein, wherein the composition has about 3 mg of the crystalline form (e.g., Form A) of the compound 6-O-(4-dimethylaminoethoxy)cinnamoyl fumagillol, free base.

In another aspect, this disclosure features a unit dose vial or pre-loaded syringe for delivering about a 1.8 mg drug product dose, which includes any one of the suspension lyophile compositions described herein, wherein the composition has about 2.25 mg of the crystalline form (e.g., Form A) of the compound 6-O-(4-dimethylaminoethoxy)cinnamoyl fumagillol, free base.

In a further aspect, this disclosure features a unit dose vial or pre-loaded syringe for about a 1.2 mg drug product dose, which includes any one of the suspension lyophile compositions described herein, wherein the composition has about 1.5 mg of the crystalline form (e.g., Form A) of the compound 6-O-(4-dimethylaminoethoxy)cinnamoyl fumagillol, free base.

In one aspect, this disclosure features a unit dose vial or pre-loaded syringe for about a 0.6 mg drug product dose, which includes any one of the suspension lyophile compositions described herein, wherein the composition has about 0.75 mg of the crystalline form (e.g., Form A) of the compound 6-O-(4-dimethylaminoethoxy)cinnamoyl fumagillol, free base.

In another aspect, this disclosure features a unit dose vial or pre-loaded syringe for delivering about a 0.3 mg drug product dose to a patient, which includes any one of the suspension lyophile compositions described herein, wherein the composition has about 0.38 mg of the crystalline form (e.g., Form A) of the compound 6-O-(4-dimethylaminoethoxy)cinnamoyl fumagillol, free base.

In one aspect, this disclosure features a reconstitution kit, which includes (i) a first container comprising a suspension lyophile composition comprising a crystalline form (e.g., Form A) of the compound 6-O-(4-dimethylaminoethoxy)cinnamoyl fumagillol, free base; and (ii) a second container comprising a diluent for the suspension lyophile composition. In some embodiments, upon reconstitution of the suspension lyophile composition with the diluent, the crystalline form (e.g., Form A) of the compound can have a cumulative size distribution at 90% of less than about 400 μm, (or less than about 200 μm) e.g., the crystalline form (e.g., Form A) of the compound can have a cumulative size distribution at 90% between about 15 μm to about 400 μm (or between about 15 and about 100 μm), between about 15 μm to about 40 μm or about 20 μm to about 40 μm, or between about 15 μm to about 30 μm, or for example, less than about 30 μm. The diluent can include water and polysorbate 80. The suspension lyophile composition can further include trehalose dehydrate. The suspension lyophile composition can further include povidone K17. The suspension lyophile composition can further include poloxamer 188.

Embodiments can also include any one or more of the following features. Compositions can include a dispersing agent (e.g., may be a non-ionic polymer (e.g., poloxamer, e.g., poloxamer 188, a tri-block copolymer having an average molecular weight of about 8400 Da), and/or a bulking agent (e.g., trehalose or hydrate thereof (e.g., trehalose dihydrate). The crystalline form (e.g., Form A) can have a particle size distribution profile suitable for use with a 23-31-gauge needle (e.g., a 27, 29 or 31-gauge needle). The composition can include substantially minimal amorphous form of 6-O-(4-dimethylaminoethoxy)cinnamoyl fumagillol, free base. The crystalline form (e.g., Form A) of the compound has a cumulative size distribution at 90% of less than about 40 μm upon reconstitution of the lyophile composition. The suspension lyophile compositions can further include a suspending agent (e.g., polyvinylpyrrolidone, e.g., povidone K18, povidone K17 or povidone K12, e.g., povidone K17). The suspension lyophile compositions can further include one or more buffer agents (e.g., the one or more buffer agents can each be independently selected from monobasic sodium phosphate, dibasic sodium phosphate and hydrates thereof; e.g., the one or more buffer agents can each be independently selected from monobasic sodium phosphate, monohydrate and dibasic sodium phosphate, heptahydrate). The suspension lyophile compositions can further include a polysorbate (e.g., polysorbate 80). The pH of the composition can be sufficient to minimize the amount of the compound in solution. The suspension lyophile compositions can have a pH of about 6.5 to about 9 at 25° C. (e.g., a pH of about 7.8 to about 8.8 at 25° C.). The suspension lyophile compositions can include about 70 to about 95 weight percent or about 78 to about 90 weigh percent of the bulking agent (e.g., about 81 to about 88 weight percent of the bulking agent, e.g., trehalose). The suspension lyophile compositions can include about 1 to about 2 weight percent of the dispersing agent. The suspension lyophile compositions can include about 0.6% to about 12% by weight of the crystalline form (e.g., Form A) of the compound.

In some embodiments, reconstituted lyophile compositions are disclosed (reconstituted using a e.g., reconstitution vehicle or medium which may comprise water and optionally an excipient), in which one (or more) of the components is/are suspended and/or dissolved in the reconstitution medium (e.g., 10% or more, 25% or more, 50% or more, 75% or more, 90% or more, 95% or more, 99% or more of the component(s) is/are suspended and/or dissolved in the reconstitution medium). Further the disclosed compositions include suspension lyophile compositions that can be filled into vials as a suspension and can also afford a suspension on lyophile reconstitution for subcutaneous injection. In some embodiments, reconstituted suspension lyophile compositions described herein (e.g., those reconstituted with a reconstitution composition that includes water, e.g., WFI, and optionally one or more pharmaceutically acceptable excipients), at least some (e.g., 10% or more, 25% or more, 50% or more, 75% or more, 90% or more, 95% or more, 96% or more, 97% or more, 98% or more, 99% or more, 99.5% or more, 99.9% or more) of the crystalline form (e.g., Form A) of 6-O-(4-dimethylaminoethoxy)cinnamoyl fumagillol, free base is suspended in the reconstitution medium. As such, therapeutically effective doses of the crystalline form can be delivered in relatively small volumes of aqueous carrier even though the crystalline form has a relatively low solubility in water at or near physiological pH. Further, disclosed compositions are substantially stable and may substantially prevent the crystalline material reverting to amorphous material. Further, in certain embodiments, the particle size of the suspended crystalline form (e.g., Form A) of 6-O-(4-dimethylaminoethoxy)cinnamoyl fumagillol, free base can be made sufficiently small (e.g., by milling, e.g., microfluidization or jet milling) so as to permit delivery of the suspension lyophile composition through relatively small gauge needles (e.g., 23-31, e.g., 27, 29, or 31 gauge), thereby enhancing patient comfort during administration. The compositions described herein can provide one or more additional advantages, such as enhanced storage stability and/or pharmacokinetic profile (e.g., a relatively low Cₘₐₓ with essentially complete systemic clearance within 36 hours of injection).

### BRIEF DESCRIPTION OF THE DRAWINGS

FIG. 1 is a graph showing particle size reduction of a ZGN-440 in 0.2% poloxamer as a function of number of passes and operating pressure (200 μm and 87 μm interaction chambers installed in series).

FIG. 2 is a graph showing correlation of particle size reduction and number of microfluidizer passes through a 200 μm interaction chamber, or a 200 μm and 87 μm interaction chamber installed in series across multiple ZGN-440 drug substance batches.

FIGS. 3A, 3B, 3C, and 3D are tables that summarize stability data for lyophile prototypes.

FIG. 4 is a graph showing vial content consistency across a full fill run (˜12,000 vials) of ZGN-440 for injectable suspension.

FIG. 5 is a graph summarizing delivery failure versus particle size for the delivery studies carried out using ZGN-440. The majority of the delivery studies were in support of the “powder-in-a-bottle” clinical product, and almost all use 2% CMC and some level of sonication.

FIG. 6 shows results from sonication and results as a function of ratio of D(90) to needle internal diameter (ID).

FIG. 7 shows PK results from a rat study.

### DETAILED DESCRIPTION

This disclosure features compositions (e.g., re-constitutable suspensions, that can be reconstituted as a suspension suitable for subcutaneous administration. The compositions include a crystalline form (e.g., Form A) of 6-O-(4-dimethylaminoethoxy)cinnamoyl fumagillol, free base and one or more pharmaceutically acceptable excipients and/or one or more pharmaceutically acceptable carriers (e.g., one or more bulking agents; one or more dispersing agents; one or more buffers; one or more suspending agents; water, e.g., water for injection (“WFI”)). Said crystalline form and the one or more pharmaceutically acceptable excipients and/or the one or more pharmaceutically acceptable carriers are sometimes referred to herein as “components” of the compositions. In some embodiments, the compositions can be in the form of a e.g., re-constitutable suspension or lyophilized cake (sometimes referred to herein as a “lyophile”). In other embodiments, the compositions can be liquid compositions, such as solutions or suspensions, e.g., a reconstituted lyophile (e.g., reconstituted with water, e.g., WFI; and optionally one or more pharmaceutically acceptable excipients).

In some embodiments, the compositions are reconstituted lyophile compositions, in which one (or more) of the components is suspended in the reconstitution medium (e.g., 10% or more, 25% or more, 50% or more, 75% or more, 90% or more, 95% or more, 99% or more of the component(s) is/are suspended in the reconstitution medium).

### Components

Crystalline form (e.g., Form A) of 6-O-(4-Dimethylaminoethoxy)Cinnamoyl Fumagillol, Free Base

The term “crystalline form” refers to a crystal form or modification that can be characterized by analytical methods such as, e.g., X-ray powder diffraction or Raman spectroscopy. The term “amorphous form” refers to a solid form that lacks the long-range order characteristic of a crystal. In some embodiments, the compositions described herein include both crystalline and amorphous forms of 6-O-(4-dimethylaminoethoxy)cinnamoyl fumagillol, free base, although the latter is typically present in relatively small amounts. In some embodiments, the compositions described herein include substantially minimal amorphous form of 6-O-(4-dimethylaminoethoxy)cinnamoyl fumagillol, free base. As used herein, “substantially minimal amorphous form of 6-O-(4-dimethylaminoethoxy)cinnamoyl fumagillol, free base” means that that the ratio (weight of crystalline form (e.g., Form A) of 6-O-(4-dimethylaminoethoxy)cinnamoyl fumagillol, free base in composition)/(total weight of 6-O-(4-dimethylaminoethoxy)cinnamoyl fumagillol, free base in composition)*100 is about 98%, or about 97%, or more than about 95%, e.g. about 95% to about 100%.

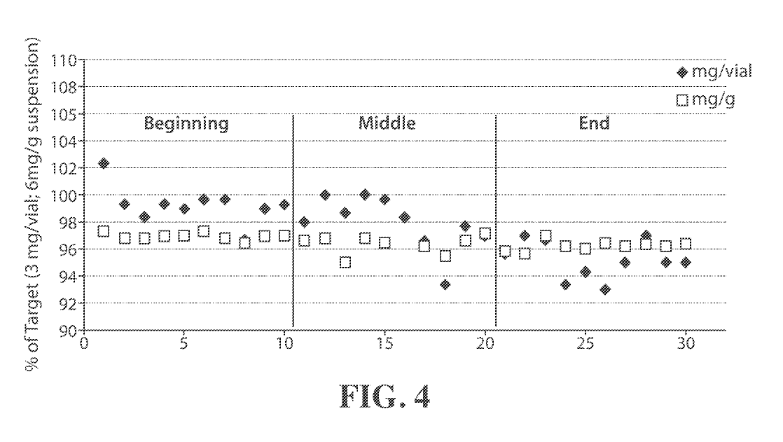

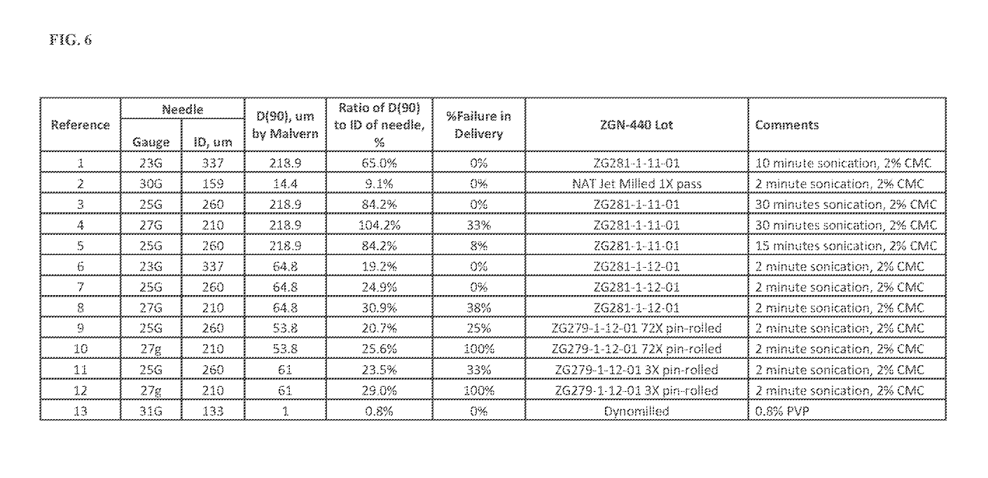

In some embodiments, the crystalline form of 6-O-(4-dimethylaminoethoxy) cinnamoyl fumagillol, free base is characterized by a powder X-ray diffraction pattern having a characteristic peak in degrees 2θ at about 13.3 (referred to herein as “Form A”, “ZGN-440”, or “beloranib”). In one embodiment, the crystalline form of 6-O-(4-dimethylaminoethoxy)cinnamoyl fumagillol (free base) is characterized by a powder X-ray diffraction pattern that has a characteristic peak in degrees 2θ at about 5.2, or is characterized by a powder X-ray diffraction pattern that has a characteristic peak in degrees 2θ at about 7.1, or is characterized by a powder X-ray diffraction pattern that has a characteristic peak in degrees 2θ at about 10.4, or is characterized by a powder X-ray diffraction pattern that has a characteristic peak in degrees 2θ at about 14.2, or is characterized by a powder X-ray diffraction pattern that has a characteristic peak in degrees 2θ at about 15.5, or is characterized by a powder X-ray diffraction pattern that has a characteristic peak in degrees 2θ at about 16.3, or is characterized by a powder X-ray diffraction pattern that has a characteristic peak in degrees 2θ at about 17.4, or is characterized by a powder X-ray diffraction pattern that has a characteristic peak in degrees 2θ at about 18.6, or is characterized by a powder X-ray diffraction pattern that has a characteristic peak in degrees 2θ at about 19.4, or is characterized by a powder X-ray diffraction pattern that has a characteristic peak in degrees 2θ at about 19.9, or is characterized by a powder X-ray diffraction pattern that has a characteristic peak in degrees 2θ at about 20.9, or is characterized by a powder X-ray diffraction pattern that has a characteristic peak in degrees 2θ at about 22.6, or is characterized by a powder X-ray diffraction pattern that has a characteristic peak in degrees 2θ at about 24.6. In another embodiment, the crystalline form is characterized by a powder X-ray diffraction pattern having at least one or more characteristic peaks in degrees 2θ at about 13.3, 17.4, and 19.9. In a further embodiment, the crystalline form is characterized by a powder X-ray diffraction pattern having at least one or more characteristic peaks in degrees 2θ at about 7.1, 13.3, 16.3, 17.4, 18.6, 19.4, and 19.9. In yet another embodiment, the crystalline form is characterized by a powder X-ray diffraction pattern having at least one or more characteristic peaks in degrees 2θ at about 5.2, 7.1, 10.4, 13.3, 14.2, 16.3, 17.4, 18.6, 19.4, and 19.9. In some embodiments, the crystalline form is characterized by a powder X-ray diffraction pattern having at least one or more characteristic peaks in degrees 2θ at about 5.2, 7.1, 10.4, 13.3, 14.2, 15.5, 16.3, 17.4, 18.6, 19.4, 19.9, 20.9, 22.6, and 24.6. The term “about” in this context means that there is an uncertainty in the measurements of the 2θ of ±0.5 (expressed in 2θ) or that there is an uncertainty in the measurements of the 2θ of ±0.2 (expressed in 2θ). For example, a contemplated crystalline form has a powder X-ray diffraction pattern shown in FIG. 4 of U.S. Pat. No. 8,349,891, U.S. Pat. No. 8,735,447, and WO 2012/064838. In one embodiment, the powder X-ray diffraction pattern of the crystalline form was obtained using Cu Kαradiation. In a further example, a contemplated crystalline form has a ¹H NMR spectrum substantially in accordance with the pattern shown in FIG. 6 of U.S. Pat. No. 8,349,891, U.S. Pat. No. 8,735,447, and WO 2012/064838, wherein the crystalline form is in solution. In still yet another example, the crystalline form of 6-O-(4-dimethylaminoethoxy)cinnamoyl fumagillol, free base has a space group of P2₁2₁2₁.

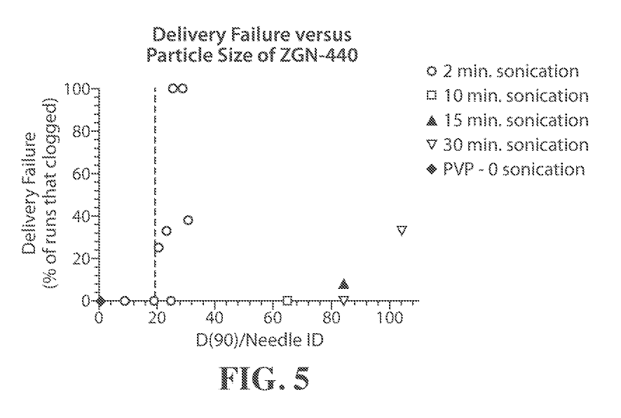

In some embodiments, the crystalline form of Form A 6-O-(4-dimethylaminoethoxy) cinnamoyl fumagillol, free base has an IR absorption spectrum having at least one or more characteristic peaks at about 2971, 2938, 2817, 2762, 1163, 1103, 832 cm⁻¹. In this context, the term “about” means that the cm⁻¹ values can vary, e.g., up to ±5 cm⁻¹. In certain embodiments, the crystalline form of Form A 6-O-(4-dimethylaminoethoxy)cinnamoyl fumagillol has IR absorption spectrum shown in FIG. 5 of U.S. Pat. No. 8,349,891, U.S. Pat. No. 8,735,447, and WO 2012/064838. In some embodiments, the crystalline form (e.g., Form A) of Form A 6-O-(4-dimethylaminoethoxy)cinnamoyl fumagillol, free base is characterized by a melting point of about 83° C., for example, and characterized by a differential scanning calorimetry profile with an endotherm at about 83.1° C. Form A, for example, has a solubility in diisopropyl ether of about 25 mg/mL at room temperature (ca. 20° C.) and about 102 mg/mL at 50° C. The solubility of Form A in solvent (e.g., an aqueous solution that may include a buffer) with a pH greater or equal to about 8.0 may be less than about 0.2 mg/mL at ca. 20° C.

In some embodiments, the crystalline form of Form A 6-O-(4-dimethylaminoethoxy) cinnamoyl fumagillol, free base is prepared by a process that includes: (a) preparing a solution of 6-O-(4-dimethylaminoethoxy)cinnamoyl fumagillol, e.g., amorphous (and/or crystalline) 6-O-(4-dimethylaminoethoxy)cinnamoyl fumagillol in a solvent (e.g., a secondary ether, toluene, n-heptane, or a combination of two or more solvents, and/or a solvent/anti-solvent system); (b) heating the solution to completely dissolve the 6-O-(4-dimethylaminoethoxy)cinnamoyl fumagillol; (c) adjusting the temperature so that solid precipitates out of the solution; and (d) isolating the crystalline form (e.g., Form A) of 6-O-(4-dimethylaminoethoxy)cinnamoyl fumagillol. In an exemplary embodiment, the secondary ether is diisopropyl ether. Other contemplated solvents include alcohols such as methanol and/or isopropanol, and solvents such as acetone, acetonitrile, cyclohexane, ethyl acetate, n-heptane, methyl ethyl ketone, methyl isobutyl ketone, tetrahydrofuran, toluene, and/or a combination of two or more thereof. For example, in one embodiment the solvent may be a toluene:n-heptane mixture, wherein the ratio of n-heptane to toluene is, for example, about 10:1, about 9:1, about 8:1, about 7:1, about 6:1, about 5:1, about 4:1, about 3:1, about 2:1, or about 1:1. In another example, the solvent or solvent/anti-solvent system is selected from ethyl acetate:n-heptane; acetone:n-heptane; or methyl ethyl ketone:n-heptane. Contemplated ratios of antisolvent to solvent include, for example, about 15:1, about 14:1, about 13:1, about 12:1, about 11:1, about 10:1, about 9:1, about 8:1, about 7:1, about 6:1, about 5:1, about 4:1, about 3:1, about 2:1, or about 1:1. In some embodiments, heating the solution comprises heating the solution to about 40° C. to about 60° C., e.g., to about 50° C. In another embodiment, adjusting the temperature comprises cooling the solution to about 0° C. to about 10° C., e.g., to about 4° C. In one embodiment, adjusting temperature comprises cooling the solution to about 4° C. or less, or to about 2° C. to about 10° C. Such systems may be used with or without seeding. For example, contemplated processes may also include incorporating or seeding a solution with an existing crystal of 6-O-(4-dimethylaminoethoxy)cinnamoyl fumagillol.

In other embodiments, the crystalline form of 6-O-(4-dimethylaminoethoxy) cinnamoyl fumagillol, free base is characterized by a powder X-ray diffraction pattern having characteristic peaks in degrees 2θ at one or more of positions at about 6.1 and 18.4 or at about 6.1, 12.2, 12.8, 12.9, 18.4, 18.6, 19.7, 20.2, 24.1, and 24.7 (sometimes referred to herein as “Form C”). The term “about” in this context means for example, that there is an uncertainty in the measurements of the 2θ of ±0.5 (expressed in 2θ) or even that there is an uncertainty in the measurements of the 2θ of ±0.2 (expressed in 2θ). For example, a contemplated crystalline form has a powder X-ray diffraction pattern shown in FIG. 14 of U.S. Pat. No. 8,349,891, U.S. Pat. No. 8,735,447, and WO 2012/064838.

In other embodiments, Form C of 6-O-(4-dimethylaminoethoxy)cinnamoyl fumagillol, free base has an IR absorption spectrum having characteristic peaks at about at least one of: 831, 894, 1106, 1159, 1249, 1287, 1512, 1602, 1631, and 1707 cm⁻¹. In this context, the term “about” means that the cm⁻¹ values can vary, e.g. up to ±5 cm⁻¹. In certain embodiments, Form C of 6-O-(4-dimethylaminoethoxy)cinnamoyl fumagillol, free base is characterized by the IR absorption spectrum shown in FIG. 15 of U.S. Pat. No. 8,349,891, U.S. Pat. No. 8,735,447, and WO 2012/064838. The contemplated crystalline Form C of 6-O-(4-dimethylaminoethoxy)cinnamoyl fumagillol, free base exhibits plate-like morphology. In one embodiment, Form C converts or reverts to Form A after, for example, about three days of storage at either 5° C. or ambient temperature. Accordingly, in certain embodiments, the compositions described herein can include Form C and/or Form A derived from Form C.

In some embodiments, the crystalline form (e.g., Form A) of 6-O-(4-dimethylaminoethoxy)cinnamoyl fumagillol, free base has a relatively small particle size (e.g., as determined on the basis of particle size distribution D values) in the compositions described herein. While not wishing to be bound by theory, it is believed that the use of smaller particle sizes can, e.g., reduce the likelihood of needle clogging when reconstituted suspension lyophile compositions are administered using smaller gauge needles (e.g., 23-31, e.g., 27, 29, or 31 gauge). In some embodiments, the particle size of the crystalline form in a lyophile is substantially maintained in the corresponding reconstituted suspension lyophile composition (e.g., the corresponding suspension lyophile composition is substantially free of aggregates or flocs of the crystalline form upon reconstitution).

In some embodiments, the crystalline form (e.g., Form A) of 6-O-(4-dimethylaminoethoxy)cinnamoyl fumagillol, free base has a cumulative size distribution at 90% (sometimes referred to herein as “D90”) of less than about 400 μm (e.g., less than about 300 μm, less than about 200 μm, less than about 100 μm, less than about 75 μm, less than about 50 μm, less than about 40 μm, less than about 30 μm, less than about 20 μm, or less than about 10 μm; e.g., less than about 30±0.5 μm, less than about 30±0.2 μm, less than about 30 μm), e.g., as measured before or after reconstitution of the lyophile composition. In some embodiments, the crystalline form (e.g., Form A) of 6-O-(4-dimethylaminoethoxy)cinnamoyl fumagillol, free base has a cumulative size distribution at 90% of between about 15 μm to about 400 μm (e.g., from between about 15 μm to about 300 μm, between about 15 μm to about 200 μm, between about 15 μm to about 100 μm, 15 μm to about 75 μm, 15 μm to about 50 μm, 15 μm to about 40 μm or about 20 μm to about 40 μm, about 15 μm to about 30 μm, about 15 μm to about 29 μm, about 20 μm to about 30 μm, about 20 μm to about 29 μm, about 25 μm to about 30 μm, or between about 25 μm to about 29 μm).

In some embodiments, a crystalline form (e.g., Form A) of 6-O-(4-dimethylaminoethoxy)cinnamoyl fumagillol, free base has a particle size distribution profile suitable for use with a 27, 29 or 31-gauge needle (e.g., 29 or 31-gauge needle, e.g., a 31-gauge needle). In some embodiments, the ratio (D90 of crystalline form)/(needle internal diameter)*100 is less than about 50% (e.g., less than about 40%, less than about 30%, less than about 25%, less than about 20%, less than about 19%, less than about 18%, less than about 17%, less than about 16%, less than about 15%, less than about 14%, less than about 13%, less than about 12%, less than about 11%, less than about 10%). In certain embodiments, the ratio (D90 of crystalline form)/(needle internal diameter)*100 is less than about 19%.

In certain embodiments, the D90 of the crystalline form is less than about 30±0.5 μm (e.g., less than about 30±0.2 μm, less than about 30 μm), and the delivery needle is 29 or 31-gauge. In certain of these embodiments, the needle internal diameter is selected so that the ratio (D90 of crystalline form)/(needle internal diameter)*100 is less than about 19%.

In other embodiments, disclosed crystalline forms of 6-O-(4-dimethylaminoethoxy)cinnamoyl fumagillol (e.g., Form A) may have a D99 of less than 20 or less than 30 microns, or a D99 that facilitates patient administration with a fine gauge needle to e.g., ensure that needle clogging would not occur with the use of e.g. 27-31 gauge needle for dose administration

In some embodiments, disclosed compositions (e.g., a reconstitutable solid or suspension, e.g., a lyophile) include from about 0.6% to about 12% (e.g., about 0.6% to about 11%, about 0.6% to about 10%, about 0.6% to about 9%, about 0.6% to about 8%, about 0.6% to about 7%, about 0.6% to about 6%, about 0.6% to about 5%, about 0.6% to about 4%, about 0.6% to about 3%; about 0.8% to about 12%, about 0.8% to about 11%, about 0.8% to about 10%, about 0.8% to about 9%, about 0.8% to about 8%, about 0.8% to about 7%, about 0.8% to about 6%, about 0.8% to about 5%, about 0.8% to about 4%, about 0.8% to about 3%; about 1% to about 12%, about 1% to about 11%, about 1% to about 10%, about 1% to about 9%, about 1% to about 8%, about 1% to about 7%, about 1% to about 6%, about 1% to about 5%, about 1% to about 4%, about 1% to about 3%; about 2% to about 12%, about 2% to about 11%, about 2% to about 10%, about 2% to about 9%, about 2% to about 8%, about 2% to about 7%, about 2% to about 6%, about 2% to about 5%, about 2% to about 4%, about 2% to about 3%) by weight of the crystalline form (e.g., Form A) of 6-O-(4-dimethylaminoethoxy)cinnamoyl fumagillol, free base.

In certain embodiments, disclosed compositions (e.g., a reconstitutable solid, e.g., a lyophile) include from about 1% to about 8% (e.g., about 2% to about 8%, about 2% to about 6%, about 1% to about 4%, about 0.8% to about 3%) by weight of the crystalline form (e.g., Form A) of 6-O-(4-dimethylaminoethoxy)cinnamoyl fumagillol, free base. For example, the compositions can include from about 2% to about 8% (e.g., about 3% to about 7%, about 4% to about 6%; e.g., 4.84%) by weight of the crystalline form. As another example, disclosed compositions can include from about 2% to about 6% (e.g., about 3% to about 5%; e.g., 3.67%) by weight of the crystalline form. As a further example, the compositions can include from about 1% to about 4% (e.g., about 2% to about 3%; e.g., 2.48%) by weight of the crystalline form. In still yet another example, disclosed compositions can include from about 0.8% to about 3% (e.g., about 0.8% to about 2%; e.g., 1.25%) by weight of the crystalline form.

In some embodiments, disclosed compositions (e.g. suspension lyophile compositions) include from about 0.6 to about 12 (e.g., about 0.6 to about 11, about 0.6 to about 10, about 0.6 to about 9, about 0.6 to about 8, about 0.6 to about 7, about 0.6 to about 6, about 0.6 to about 5, about 0.6 to about 4, about 0.6 to about 3; about 0.8 to about 12, about 0.8 to about 11, about 0.8 to about 10, about 0.8 to about 9, about 0.8 to about 8, about 0.8 to about 7, about 0.8 to about 6, about 0.8 to about 5, about 0.8 to about 4, 0.8 to about 3; 1 to about 12, about 1 to about 11, about 1 to about 10, about 1 to about 9, about 1 to about 8, about 1 to about 7, about 1 to about 6, about 1 to about 5, about 1 to about 4, 1 to about 3; 2 to about 12, about 2 to about 11, about 2 to about 10, about 2 to about 9, about 2 to about 8, about 2 to about 7, about 2 to about 6, about 2 to about 5, about 2 to about 4, about 2 to about 3) mg/mL of the crystalline form (e.g., Form A) of 6-O-(4-dimethylaminoethoxy)cinnamoyl fumagillol, free base.

In some embodiments, disclosed compositions (e.g., suspension lyophile compositions) include from about 1 to about 8 (e.g., about 2 to about 8, about 2 to about 6, about 1 to about 4, about 0.6 to about 3) mg/mL of the crystalline form (e.g., Form A) of 6-O-(4-dimethylaminoethoxy)cinnamoyl fumagillol, free base. For example, disclosed compositions can include from about 2 to about 8 (e.g., about 3 to about 7, about 4 to about 6; e.g., 4.00) mg/mL of the crystalline form. As another example, disclosed compositions can include from about 2 to about 6 (e.g., about 2% to about 4; e.g., 3.00) mg/mL of the crystalline form. As a further example, the compositions can include from about 1 to about 4 (e.g., about 1 to about 3; e.g., 2.00) mg/mL of the crystalline form. In still yet another example, the compositions can include from about 0.6 to about 3 (e.g., about 0.6% to about 2%; e.g., 1.00) mg/mL of the crystalline form.

In some embodiments, disclosed compositions (e.g. lyophile compositions or reconstituted compositions) include from about 0.1 mg to about 10 mg (e.g., about 0.1 to about 7.5 mg, about 0.1 to about 5 mg, 0.1 to about 3 mg; e.g., 0.3 mg, 0.38 mg, 0.4 mg, 0.5 mg, 0.75 mg, 0.6 mg, 0.75 mg, 1 mg, 1.5 mg, 2 mg, 2.25 mg, 1.5 mg, 1.2 mg, 1.8 mg, 1.8 mg, 2.4 mg, 2.5 mg, 3.0 mg, 3.5 mg, 4 mg, 5 mg, 5.6 mg, 6 mg, 7 mg, 8, mg) of the crystalline form (e.g., Form A) of 6-O-(4-dimethylaminoethoxy)cinnamoyl fumagillol, free base.

In some embodiments, disclosed compositions (e.g. lyophile compositions or reconstituted compositions) provide a dose of from about 0.1 mg to about 10 mg (e.g., about 0.1 to about 7.5 mg, about 0.1 to about 5 mg, 0.1 to about 3 mg; e.g., 0.3 mg, 0.5 mg, 0.6 mg, 0.75 mg, 1 mg, 2 mg, 2.25 mg, 1.5 mg, 1.2 mg, 1.8 mg, 1.8 mg, 2.4 mg, 2.5 mg, 3.0 mg, 3.2 mg, 4 mg, 4.5 mg, 5 mg or even 6 mg) of the crystalline form (e.g., Form A) of 6-O-(4-dimethylaminoethoxy)cinnamoyl fumagillol, free base.

In certain embodiments, the amount of the crystalline form (e.g., Form A) of 6-O-(4-dimethylaminoethoxy)cinnamoyl fumagillol, free base that is present in the compositions is greater than the desired delivery dose. For example, compositions intended to deliver, e.g., a dose of 6 mg, 4.5 mg, 3.2 mg, 2.8 mg, 2.4 mg, 1.8 mg, 1.2 mg, 0.6 mg, or 0.3 mg of the crystalline form can include 7 mg, 5.6 mg, 4 mg, 3.5 mg, 3 mg, 2.25 mg, 1.5 mg, 0.75 mg, and 0.38 mg, respectively, of the crystalline form.

In some embodiments, disclosed lyophile compositions (i) include from about 2% to about 8% (e.g., about 3% to about 7%, about 4% to about 6%; e.g., 4.84%) by weight of the crystalline form (e.g., Form A) of 6-O-(4-dimethylaminoethoxy)cinnamoyl fumagillol, free base; or (ii) include from about 2 to about 8 (e.g., about 3 to about 7, about 4 to about 6; e.g., 4.00) mg/mL of the crystalline form; and/or (iii) include from about 0.1 to about 10 mg (e.g., 7 mg, 5.6 mg, 4 mg, 3.5 mg, 3 mg) of the crystalline form; and (iv) provide a dose of from about 0.1 mg to about 5 mg (e.g., 6 mg, 4.5 mg, 3.2 mg, 2.8 mg, 2.4 mg) of the crystalline form. In certain embodiments, the D90 of the crystalline form is less than about 40 μm (e.g., less than about 30±0.5 μm, less than about 30±0.2 μm, less than about 30 μm).

In some embodiments, disclosed lyophile compositions (i) include from about 2% to about 6% (e.g., about 3% to about 5%; e.g., 3.67%) by weight of the crystalline form (e.g., Form A) of 6-O-(4-dimethylaminoethoxy)cinnamoyl fumagillol, free base; or (ii) include from about 2 to about 6 (e.g., about 2 to about 4; e.g., 3.00) mg/mL of the crystalline form; and/or (iii) include from about 0.1 to about 5 mg (e.g., 2.25 mg) of the crystalline form; and (iv) provide a dose of from about 0.1 mg to about 5 mg (e.g., 1.8 mg) of the crystalline form. In certain embodiments, the D90 of the crystalline form is less than about 40 μm (e.g., less than about 30±0.5 μm, less than about 30±0.2 μm, less than about 30 μm).

In some embodiments, the disclosed lyophile compositions (i) include from about 1% to about 4% (e.g., about 2% to about 3%; e.g., 2.48%) by weight of the crystalline form (e.g., Form A) of 6-O-(4-dimethylaminoethoxy)cinnamoyl fumagillol, free base; or (ii) include from about 1 to about 4 (e.g., about 1 to about 3; e.g., 2.00) mg/mL of the crystalline form; and (iii) include from about 0.1 to about 3 mg (e.g., 1.5 mg) of the crystalline form; and (iv) provide a dose of from about 0.1 mg to about 5 mg (e.g., 1.2 mg) of the crystalline form. In certain embodiments, the D90 of the crystalline form is less than about 40 μm (e.g., less than about 30±0.5 μm, less than about 30±0.2 μm, less than about 30 μm).

In some embodiments, disclosed lyophile compositions (i) include from about 0.8% to about 3% (e.g., about 0.8% to about 2%; e.g., 1.25%) by weight of the crystalline form (e.g., Form A) of 6-O-(4-dimethylaminoethoxy)cinnamoyl fumagillol, free base; or (ii) include from about 0.6 to about 3 (e.g., about 0.6% to about 2%; e.g., 1.00) mg/mL of the crystalline form; and (iii) include from about 0.1 to about 3 mg (e.g., 0.75 mg, 0.38 mg) of the crystalline form; and (iv) provide a dose of from about 0.1 mg to about 3 mg (e.g., 0.6 mg, 0.3 mg) of the crystalline form. In certain embodiments, the D90 of the crystalline form is less than about 40 μm (e.g., less than about 30±0.5 μm, less than about 30±0.2 μm, less than about 30 μm).

Disclosed compositions, in certain embodiments, are suspension lyophile compositions (or are lyophiles that provide suspension lyophile compositions upon reconstitution), in which at least some (e.g., 10% or more, 25% or more, 50% or more, 75% or more, 90% or more, 95% or more, 96% or more, 97% or more, 98% or more, 99% or more, 99.5% or more, 99.9% or more) of the crystalline form (e.g., Form A) of 6-O-(4-dimethylaminoethoxy)cinnamoyl fumagillol, free base is suspended in the reconstitution medium.

Various contemplated excipients that may be included in a disclosed composition alone or in combination are described in the sections below, which are organized by function. For purposes of clarification, any excipient described herein may be performing one or more functions in addition to, or altogether different from, those delineated below; and therefore is not limited to the particular associated function delineated below.

In some embodiments, disclosed lyophile compositions include one or more dispersing agents. Contemplated dispersing agents include non-ionic polymers (e.g., a poloxamer, e.g., poloxamer 188; a polysorbate, e.g., polysorbate 80, polysorbate 40, polysorbate 20), non-ionic surfactants (e.g., a Span, e.g., Span 85, Span 80, Span 40, Span 20; Solutol H15; Cremophor EL) and/or lecithin. In certain embodiments, the one or more dispersing agents is a non-ionic polymer (e.g., a poloxamer, e.g., poloxamer 188). In some embodiments, the compositions include from about 0.5% to about 5% (e.g., about 2% to about 5%, about 1% to about 3%; about 1% to about 2%, e.g., about 2.15% or about 2.02%) by weight of total poloxamer. In some embodiments, the compositions include from about 0.5 to about 5 (e.g., about 2 to about 5, about 1 to about 3; about 1 to about 2) mg/mL of total poloxamer. In certain embodiments, poloxamer 188 may be introduced, for example, prior to other excipients in certain intermediary processing steps for making the compositions described herein; e.g., as a wetting agent or disbursement agent in the microfluidization processes used to reduce particle size of the crystalline form (e.g., Form A) of 6-O-(4-dimethylaminoethoxy)cinnamoyl fumagillol, free base. In some embodiments, a dispersing agent is selected that is suitable for injection

In other embodiments, a disclosed lyophile suspension such as described above also includes a dispersing agent such as a polysorbate (e.g. polysorbate 80, polysorbate 40, polysorbate 20) dispersing agent (e.g., the dispersing agent is present in the lyophile prior to reconstitution with a diluent). In some embodiments, disclosed lyophile compositions optionally include from about 0.5% to about 8% (e.g., about 1% to about 6%, e.g., 5.99%) of a polysorbate. Contemplated dispersing agents may include one or more of e.g., Span 85, Span 80, Span 40, Span 20), e.g. optionally include about 0.5% to about 8% (e.g., about 1% to about 6%, e.g., 5.99%) of Span, and/or may include a dispersing agent such as lecithin, e.g. may optionally include about 0.5% to about 8% (e.g., about 1% to about 6%, e.g., 5.99%) of lecithin.

In other embodiments, a disclosed lyophile suspension optionally includes Solutol H15 as a dispersing agent prior to reconstitution. In some embodiments, disclosed lyophile compositions optionally include from about 0.5% to about 8% (e.g., about 1% to about 6%, e.g., 5.99%) of Solutol H15. In certain embodiments, Solutol H15 is included in the lyophile composition prior to reconstitution.

In other embodiments, a disclosed lyophile suspension optionally includes Cremophor EL as a dispersing agent prior to reconstitution. In some embodiments, disclosed lyophile compositions optionally include from about 0.5% to about 8% (e.g., about 1% to about 6%, e.g., 5.99%) of Cremophor EL. In certain embodiments, Cremophor EL is included in the lyophile composition prior to reconstitution.

Disclosed lyophile composition can include one or more bulking agents, e.g. in addition to a dispersing agent(s) such as one or more described above. Contemplated compositions may include bulking agents such as sugars (e.g., trehalose, trehalose dihydrate, mannitol, lactose, raffinose or sucrose or any combination thereof). In some embodiments, disclosed compositions may include from about 50% to about 95% (e.g., about 70% to about 95%, e.g., 79.3%; about 81% to about 88%, e.g., 84.37%) by weight of the one or more bulking agents, e.g. trehalose, trehalose dihydrate, mannitol, lactose, raffinose or sucrose or any combination thereof. In some embodiments, the compositions include from about 50 to about 90 (e.g., about 60 to about 80; about 65 to about 75) mg/mL of the one or more bulking agents, e.g., trehalose, tehalose dehydrate, mannitol, lactose, raffinose, or sucrose or any combination thereof. In some embodiments, the weight percent of bulking agent is selected for manufacturability. for example, a lower weight percent of bulking agent may result in a less advantageous lyophile cake.

For example, disclosed lyophile compositions may include a weight percent of bulking agent (e.g., trehalose dihydrate, mannitol, lactose, raffinose or sucrose or any combination thereof) that provides an iso-osmotic suspension upon reconstitution. In certain embodiments, trehalose and/or trehalose dihydrate is used as a bulking agent in a disclosed suspension lyophile composition or lyophilized drug product and/or as a tonicity modifier in the reconstituted suspension. At least in some embodiments, lyophile stability in lyophile compositions having mannitol—as compared to e.g., trehalose, may have significantly inferior stability. In some embodiments, the one or more bulking agents in a disclosed lyophile composition include sucrose. In other embodiments, the one or more bulking agents in a disclosed lyophile composition include raffinose. In further embodiments, the one or more bulking agents in a disclosed lyophile composition include lactose.

In some embodiments, disclosed lyophile compositions optionally may include one or more suspending agents in addition to a bulking agent and/or a dispersing agent. Contemplated suspending agents include polyethylene glycol (“PEG”, e.g., PEG 3350 and/or PEG 4000), polyvinylpyrrolidinone (“PVP”, e.g., Povidone, e.g. K-12, K-17, K-18, K-25, K-29/32; e.g., K-12 or K-17), sodium carboxymethyl cellulose, or methylcellulose or any combination thereof.

For example, in certain embodiments the one or more suspending agents in the lyophile composition include polyvinylpyrrolidinone (“PVP”), e.g., Povidone, e.g. K-12, K-17, K-18, K-25, K-29/32; e.g., K-12 or K-17. In some embodiments, disclosed lyophile compositions include from about 1% to about 10% (e.g., about 1% to about 8%; about 3% to about 7%; about 4% to about 6%, e.g., 4.86% or 5.17%) by weight of PVP. In some embodiments, disclosed lyophile compositions include from about 1 to about 10 (e.g., about 2 to about 6; about 3 to about 5) mg/mL of PVP. In certain embodiments, disclosed lyophiles compositions may include PVP, that can have improved stability (e g, minimal or significantly less formation of a dimer degradant compared with lyophiles containing PEG 3350).

In other embodiments, the one or more suspending agents in a disclosed lyophile composition, if present, may include polyethylene glycol (“PEG”, e.g., PEG 3350 and/or PEG 4000), for example, disclosed lyophile compositions may include from about 1% to about 10% (e.g., about 1% to about 8%; about 3% to about 7%; about 4% to about 6%, e.g., 4.86% or 5.17%) by weight of PEG. In some embodiments, disclosed lyophile compositions include from about 1 to about 10 (e.g., about 2 to about 6; about 3 to about 5) mg/mL of PEG.

In certain embodiments, the one or more suspending agents in a disclosed lyophile composition may include sodium carboxymethyl cellulose. In some embodiments, disclosed lyophile compositions include from about 1% to about 10% (e.g., about 1% to about 8%; about 3% to about 7%; about 4% to about 6%, e.g., 4.86% or 5.17%) by weight of sodium carboxymethyl cellulose. In some embodiments, disclosed lyophile compositions include from about 1 to about 10 (e.g., about 2 to about 6; about 3 to about 5) mg/mL of sodium carboxymethyl cellulose.

In certain embodiments, the one or more suspending agents in a disclosed lyophile composition include methylcellulose. In some embodiments, disclosed lyophile compositions include from about 1% to about 10% (e.g., about 1% to about 8%; about 3% to about 7%; about 4% to about 6%, e.g., 4.86% or 5.17%) by weight of methylcellulose. In some embodiments, disclosed lyophile compositions include from about 1 to about 10 (e.g., about 2 to about 6; about 3 to about 5) mg/mL of methylcellulose.

Disclosed compositions can include one or more buffer agents, for example, one or more (e.g., two) phosphate buffers may be included in the disclosed contemplated lyophile compositions. Contemplated phosphate buffer agents include dibasic sodium phosphate, monobasic sodium phosphate, dibasic potassium phosphate, or monobasic potassium phosphate, or any combination thereof. For example, one or more buffer agents can be used to adjust and maintain the pH in the reconstituted product, and in some embodiments, may be incorporated into a disclosed lyophile before lyophilization. Contemplated lyophilized compositions may have a pH of about 6.5 to about 9, or about 7 to about 9, or about 7.4 to about 9 (e.g., at 25° C.). In some embodiments, the buffered concentrations in reconstituted product may be about 10 to 50 millimolar. For example, a phosphate buffer may be included so that the concentration is e.g., about 15 mM upon reconstitution. In an embodiment, the phosphate buffer and/or e.g. the amount of phosphate buffer may be selected to achieve a pH of the lyophile composition (e.g., about 6.5 to about 9, about 7 to about 9, about 7.4 to about 9, about 7.8 to about 8.3, about 7.8 to about 8.8, for example, about 8.3 plus or minus 0.5) at 25° C. For example, the pH of a disclosed lyophile composition and/or disclosed reconstituted composition is selected to minimize soluble ZGN-440, for example, to minimize soluble forms of the compound. In some embodiments, the pH of a disclosed lyophile may be achieved by use of the selected ratio of two or more phosphate salt forms, e.g., disodium, monosodium, dipotassium, monopotassium, or any combination of two or more thereof. For example, a buffer strength of about 23 mM a fill lyophile suspension (e.g., about 15 mM upon reconstitution) is contemplated. In some embodiments, phosphate buffers may be hydrate forms that e.g., may minimize potential moisture uptake during excipient dispensing.

In some embodiments, disclosed lyophile compositions include from about 0.5% to about 8% (e.g., about 1 to about 10%, about 3% to about 7%; or about 4% to about 6%) by weight of total phosphate buffer. In some embodiments, disclosed lyophile compositions include from about 0.5 to about 8 (e.g., about 2 to about 6; about 3 to about 5) mg/mL of total phosphate buffer. In some embodiments, disclosed lyophile compositions include from about 0.5% to about 8% (e.g., about 1% to about 6%; about 2% to about 4%, for example 2.80% or 2.63%) by weight of a first (e.g. phosphate) buffer and about 0.01% to about 5% (e.g., 0.113% or 0.106%) of a second phosphate buffer. In some embodiments, the compositions include from about 0.5 to about 8 (e.g., about 2 to about 6; about 3 to about 5) mg/mL of a first phosplate buffer and about 0.01 to about 5 mg/mL of a second phosphate buffer.

In other embodiments, disclosed lyophile compositions may include one or more organic buffers that may include organic small molecules, e.g., amino acids and salts thereof, amino sugars and salts thereof or amine bases and salts thereof. For example, contemplated organic buffers may include glutamic acid and salts thereof, e.g., in some embodiments, a disclosed lyophile composition may include from about 1% to about 9% by weight of glutamic acid and/or salts thereof. In other embodiments, the one or more organic buffers in a disclosed lyophile composition include glycine and salts thereof. In some embodiments, disclosed lyophile compositions include from about 0.5% to about 5% by weight of glycine and salts thereof. In further embodiments, the one or more organic buffers in a disclosed lyophile composition include arginine and salts thereof. In some embodiments, disclosed lyophile compositions include from about 1% to about 11% by weight of glycine and salts thereof. In certain embodiments, the one or more organic buffers in a disclosed lyophile composition include meglumine and salts thereof. In some embodiments, disclosed lyophile compositions include from about 2% to about 12% by weight of meglumine and salts thereof. In certain embodiments, the one or more organic buffers in a disclosed lyophile composition include tromethamine and salts thereof. In some embodiments, disclosed lyophile compositions include from about 1% to about 8% by weight of tromethamine and salts thereof. In certain embodiments, the one or more organic buffers in a disclosed lyophile composition include trolamine and salts thereof. In some embodiments, disclosed lyophile compositions include from about 1% to about 9% by weight of trolamine and salts thereof.

In some embodiments, a disclosed composition may include WFI, e.g. that may be used as a solvent/diluent during compounding or reconstitution. In an embodiment, WFI is used as a processing aid during microfluidization and lyophilization, but the majority of the WFI may be removed during the lyophilization process creating the lyophile suspension. In certain embodiments, a reconstitution composition consists essentially of water. In certain other embodiments, a reconstitution composition comprises WFI and optionally polysorbate 80. In certain embodiments, polysorbate 80 is included in the WFI reconstitution medium.

In each of the foregoing embodiments, disclosed compositions may be suspension lyophile compositions (or are lyophiles that provide compositions upon reconstitution), in which at least some (e.g., 10% or more, 25% or more, 50% or more, 75% or more, 90% or more, 95% or more, 96% or more, 97% or more, 98% or more, 99% or more, 99.5% or more, 99.9% or more) of the crystalline form (e.g., Form A) of 6-O-(4-dimethylaminoethoxy)cinnamoyl fumagillol, free base is suspended in the reconstitution medium.

In some embodiments, disclosed compositions (i) include from about 2% to about 8% (e.g., about 3% to about 7%, about 4% to about 6%; e.g., 4.84%) by weight of the crystalline form (e.g., Form A) of 6-O-(4-dimethylaminoethoxy)cinnamoyl fumagillol, free base; or (ii) include from about 2 to about 8 (e.g., about 3 to about 7, about 4 to about 6; e.g., 4.00) mg/mL of the crystalline form; or (iii) include from about 0.1 to about 10 mg (e.g., 7 mg, 5.6 mg, 4 mg, 3.5 mg, 3 mg) of the crystalline form; or (iv) provide a dose of from about 0.1 mg to about 5 mg (e.g., 6 mg, 4.5 mg, 3.2 mg, 2.8 mg, 2.4 mg) of the crystalline form. In certain embodiments, the D90 of the crystalline form present in a disclosed composition (either lyophile composition or reconstituted composition) is less than about 40 μm (e.g., less than about 30±0.5 μm, less than about 30±0.2 μm, less than about 30 μm). Disclosed compositions can further include one or more of a component independently selected for each occurrence of a component in (a)-(f) below (i.e. independently selected from one or more of components in (a)-(f): (a) from about 0.5% to about 5% (e.g., about 1 to 10%, about 2% to about 5%, about 1% to about 3%, about 1% to about 2%, about 3% to about 7%; or about 4% to about 6%, e.g., about 2.15% or 2.02%) by weight, or from about 0.5 to about 5 (e.g., about 2 to about 5, about 1 to about 3, about 1 to about 2) mg/mL, of one or more dispersing agents (e.g., a non-ionic polymer, e.g., a poloxamer, e.g., poloxamer 188); (b) from about 70% to about 95% (e.g., about 81% to about 88%, e.g., about 85% or about 79%) by weight, or from about 60 to about 80 (e.g., about 65 to about 75) mg/mL, of one or more bulking agents (e.g., trehalose, trehalose dihydrate, mannitol, sucrose, raffinose, and/or lactose); (c) from about 1% to about 10% (e.g., about 1% to about 8%, e.g., 5.17% or 4.86%) by weight, or from about 2 to about 6 (e.g., about 3 to about 5) mg/mL, of one or more suspending agents (e.g., PVP, e.g., K-32, K-29, K-18, K-17 or K-12; PEG, e.g, PEG 3350 or PEG 4000; sodium carboxymethyl cellulose; methylcellulose); (d) from about 0.5% to about 8% (e.g., about 3% to about 7%; about 4% to about 6%) by weight, or about 1 to about 10 (e.g., about 2 to about 6; about 3 to about 5) mg/mL, of total buffer (e.g., phosphate buffer, e.g., four phosphate salt forms, e.g., disodium, monosodium, dipotassium, monopotassium); (e) from about 0.5% to about 8% (e.g., about 1% to about 6%, e.g., 5.99%) by weight of optionally included polysorbate (e.g., polysorbate 80, polysorbate 40, polysorbate 20), Span (e.g., Span 85, Span 80, Span 40, Span 20), lecithin, Solutol H15, Cremophor EL; and (f) WFI (optionally including Polysorbate 80).

In some embodiments, disclosed compositions (i) include from about 2% to about 6% (e.g., about 3% to about 5%; e.g., 3.67%) by weight of the crystalline form (e.g., Form A) of 6-O-(4-dimethylaminoethoxy)cinnamoyl fumagillol, free base; or (ii) include from about 2 to about 6 (e.g., about 2 to about 4; e.g., 3.00) mg/mL of the crystalline form; or (iii) include from about 0.1 to about 5 mg (e.g., 2.25 mg) of the crystalline form; or (iv) provide a dose of from about 0.1 mg to about 5 mg (e.g., 1.8 mg) of the crystalline form. In certain embodiments, the D90 of the crystalline form is less than about 40 μm (e.g., less than about 30±0.5 μm, less than about 30±0.2 μm, less than about 30 μm). Disclosed compositions can further include one or more of a component independently selected for each occurrence of a component in (a)-(f) below (i.e. independently selected from one or more of components in (a)-(f): (a) from about 0.5% to about 5% (e.g., about 2% to about 5%, about 1% to about 3%, about 1% to about 2%) by weight, or from about 0.5 to about 5 (e.g., about 2 to about 5, about 1 to about 3, e.g., 2.15% or 2.02%; about 1 to about 2) mg/mL, of one or more dispersing agents (e.g., a non-ionic polymer, e.g., a poloxamer, e.g., poloxamer 188); (b) from about 70% to about 95% (e.g., about 81% to about 88%) by weight, or from about 60 to about 80 (e.g., about 65 to about 75) mg/mL, of one or more bulking agents (e.g., trehalose, trehalose dyhydrate, mannitol, sucrose, raffinose, lactose); (c) from about 1% to about 10% (e.g., about 1% to about 8%, e.g., 5.17% or 4.86%) by weight, or from about 2 to about 6 (e.g., about 3 to about 5) mg/mL, of one or more suspending agents (e.g., PVP, e.g., K-32, K-29, K-18, K-17 or K-12; PEG, e.g, PEG 3350 or PEG 4000; sodium carboxymethyl cellulose; methylcellulose); (d) from about 0.5% to about 8% (e.g., about 3% to about 7%; about 4% to about 6%) by weight, or about 1 to about 10 (e.g., about 2 to about 6; about 3 to about 5) mg/mL, of total buffer (e.g., phosphate buffer, e.g., four phosphate salt forms, e.g., disodium, monosodium, dipotassium, monopotassium); (e) from about 0.5% to about 8% (e.g., about 1% to about 6%, e.g., 5.99%) by weight of optionally included polysorbate (e.g., polysorbate 80, polysorbate 40, polysorbate 20), Span (e.g., Span 85, Span 80, Span 40, Span 20), lecithin, Solutol H15, Cremophor EL; and (f) WFI (optionally including Polysorbate 80).

In some embodiments, disclosed compositions (i) include from about 1% to about 4% (e.g., about 2% to about 3%; e.g., 2.48%) by weight of the crystalline form (e.g., Form A) of 6-O-(4-dimethylaminoethoxy)cinnamoyl fumagillol, free base; or (ii) include from about 1 to about 4 (e.g., about 1 to about 3; e.g., 2.00) mg/mL of the crystalline form; or (iii) include from about 0.1 to about 3 mg (e.g., 1.5 mg) of the crystalline form; or (iv) provide a dose of from about 0.1 mg to about 5 mg (e.g., 1.2 mg) of the crystalline form. In certain embodiments, the D90 of the crystalline form is less than about 40 μm (e.g., less than about 30±0.5 μm, less than about 30±0.2 μm, less than about 30 μm). Disclosed compositions can further include one or more of a component independently selected for each occurrence of a component in (a)-(f) below (i.e. independently selected from one or more of components in (a)-(f): (a) from about 0.5% to about 5% (e.g., about 2% to about 5%, about 1% to about 3%, about 1% to about 2%) by weight, or from about 0.5 to about 5 (e.g., about 2 to about 5, about 1 to about 3, e.g., 2.15% or 2.02%; about 1 to about 2) mg/mL, of one or more dispersing agents (e.g., a non-ionic polymer, e.g., a poloxamer, e.g., poloxamer 188); (b) from about 70% to about 95% (e.g., about 81% to about 88%) by weight, or from about 60 to about 80 (e.g., about 65 to about 75) mg/mL, of one or more bulking agents (e.g., trehalose, trehalose dyhydrate, mannitol, sucrose, raffinose, lactose); (c) from about 1% to about 10% (e.g., about 1% to about 8%, e.g., 5.17% or 4.86%) by weight, or from about 2 to about 6 (e.g., about 3 to about 5) mg/mL, of one or more suspending agents (e.g., PVP, e.g., K-32, K-29, K-18, K-17 or K-12; PEG, e.g, PEG 3350 or PEG 4000; sodium carboxymethyl cellulose; methylcellulose); (d) from about 0.5% to about 8% (e.g., about 3% to about 7%; about 4% to about 6%) by weight, or about 1 to about 10 (e.g., about 2 to about 6; about 3 to about 5) mg/mL, of total buffer (e.g., phosphate buffer, e.g., four phosphate salt forms, e.g., disodium, monosodium, dipotassium, monopotassium); (e) from about 0.5% to about 8% (e.g., about 1% to about 6%, e.g., 5.99%) by weight of optionally included polysorbate (e.g., polysorbate 80, polysorbate 40, polysorbate 20), Span (e.g., Span 85, Span 80, Span 40, Span 20), lecithin, Solutol H15, Cremophor EL; and (f) WFI (optionally including Polysorbate 80).

In some embodiments, disclosed compositions (i) include from about 0.8% to about 3% (e.g., about 0.8% to about 2%; e.g., 1.25%) by weight of the crystalline form (e.g., Form A) of 6-O-(4-dimethylaminoethoxy)cinnamoyl fumagillol, free base; or (ii) include from about 0.6 to about 3 (e.g., about 0.6% to about 2%; e.g., 1.00) mg/mL of the crystalline form; or (iii) include from about 0.1 to about 3 mg (e.g., 0.75 mg, 0.38 mg) of the crystalline form; or (iv) provide a dose of from about 0.1 mg to about 5 mg (e.g., 0.6 mg, 0.3 mg) of the crystalline form. In certain embodiments, the D90 of the crystalline form is less than about 40 μm (e.g., less than about 30±0.5 μm, less than about 30±0.2 μm, less than about 30 μm). Disclosed compositions can further include one or more of a component independently selected for each occurrence of a component in (a)-(f) below (i.e. independently selected from one or more of components in (a)-(f): (a) from about 0.5% to about 5% (e.g., about 2% to about 5%, about 1% to about 3%, about 1% to about 2%) by weight, or from about 0.5 to about 5 (e.g., about 2 to about 5, about 1 to about 3, e.g., 2.15% or 2.02%; about 1 to about 2) mg/mL, of one or more dispersing agents (e.g., a non-ionic polymer, e.g., a poloxamer, e.g., poloxamer 188); (b) from about 70% to about 95% (e.g., about 81% to about 88%) by weight, or from about 60 to about 80 (e.g., about 65 to about 75) mg/mL, of one or more bulking agents (e.g., trehalose, trehalose dyhydrate, mannitol, sucrose, raffinose, lactose); (c) from about 1% to about 10% (e.g., about 1% to about 8%, e.g., 5.17% or 4.86%) by weight, or from about 2 to about 6 (e.g., about 3 to about 5) mg/mL, of one or more suspending agents (e.g., PVP, e.g., K-32, K-29, K-18, K-17 or K-12; PEG, e.g, PEG 3350 or PEG 4000; sodium carboxymethyl cellulose; methylcellulose); (d) from about 0.5% to about 8% (e.g., about 3% to about 7%; about 4% to about 6%) by weight, or about 1 to about 10 (e.g., about 2 to about 6; about 3 to about 5) mg/mL, of total buffer (e.g., phosphate buffer, e.g., four phosphate salt forms, e.g., disodium, monosodium, dipotassium, monopotassium); (e) from about 0.5% to about 8% (e.g., about 1% to about 6%, e.g., 5.99%) by weight of optionally included polysorbate (e.g., polysorbate 80, polysorbate 40, polysorbate 20), Span (e.g., Span 85, Span 80, Span 40, Span 20), lecithin, Solutol H15, Cremophor EL; and (f) WFI (optionally including Polysorbate 80).

For example, a disclosed suspension lyophile may have exemplary components such as shown in Table AA (e.g., for a vial that includes about 3 mg of ZGN-440; all % are weight percent):

TABLE AA

| Product A | Product B |
|---|---|
| pH: 6.5-9, (or e.g., 7.8-8.8 or about 8.3) at RT | pH: 6.5-9, (or e.g., 7.8-8.8 or about 8.3) at RT |
| Bulking agent: about 70% to about 95% in | Bulking agent: about 70% to about |
| anhydrous lyophile (e.g., about 81% to about | 95% in anhydrous lyophile (e.g., |
| 88%, or about 85%) and selected from anhydrous | about 81% to about 88%, or about 79 |
| trehalose; anhydrous dehydrate, mannitol, | or 80% and selected from anhydrous |
| sucrose, lactose and/or raffinose | trehalose; anhydrous dehydrate, |
| | mannitol, sucrose, lactose and/or |
| | raffinose |
| Dispersing Agent: about 0.5 to about 5%, or 2 to | Dispersing Agent: |
| 5% (e.g., about 2.15% in anhydrous lyophile) of | about 0.5 to about 5%, or 2 to 5% |
| poloxamer 188 | (e.g., about 2.02% in anhydrous |
| | lyophile) of poloxamer 188 and |
| | about 0.5 to about 8% (e.g., about 1- |
| | 6%, or about 6% (e.g., about 4 mg) of |
| | polysorbate 80, polysorbate 20, |
| | polysorbate 40, Span 20, 40, 80, 85; |
| | lecithin, solutol H15, and/or cremaphor |
| | EL. |
| Suspending agent: about 1-10%, (e.g. about 1-8%, | Suspending agent: about 1-10%, (e.g. |
| or about 5.17% in anhydrous lyophile) of PVP, | about 1-8%, or about 4.86% in |
| PVP K17, K18, K12, K29, K32, Sodium CMC, | anhydrous lyophile) of PVP, PVP |
| methylcellulose, PEG 3350 and/or PEG4000) | K17, K18, K12, K29, K32, Sodium |
| | CMC, methylcellulose, PEG 3350 |
| | and/or PEG4000) |

In each of the foregoing embodiments, compositions are suspension lyophile compositions (or are lyophiles that provide suspension lyophile compositions upon reconstitution), in which at least some (e.g., 10% or more, 25% or more, 50% or more, 75% or more, 90% or more, 95% or more, 96% or more, 97% or more, 98% or more, 99% or more, 99.5% or more, 99.9% or more) of the crystalline form (e.g., Form A) of 6-O-(4-dimethylaminoethoxy)cinnamoyl fumagillol, free base is suspended in the reconstitution medium.

### Methods of Treatment

In certain embodiments, the disclosure provides a method of treating and or ameliorating obesity in a patient in need thereof by administering a composition described herein. Also provided herein are methods for inducing weight loss in a patient in need thereof, comprising administering a composition described herein. For example, contemplated herein is a method of treating Prader-Willi syndrome comprising administering a disclosed composition.

Other contemplated methods of treatment include methods of treating or amelioriating an obesity-related condition or co-morbidity, by administering a composition described herein to a subject. For example, contemplated herein are methods for treating type 2 diabetes in a patient in need thereof and/or method of treating a patient suffering from diabetes, for other contemplated diseases or disorders

In particular, in certain embodiments, the disclosure provides a method of treating the above medical indications comprising administering to a subject in need thereof a composition described herein.

The compositions described herein may provide a patient with a body weight loss of about 0.3% to about 2%, about 0.4% to about 2%, or about 0.5% to about 2% or more, or about 0.5 kg to about 2 kg or more of the initial patient weight even after an initial dose, or after administration of two doses, or after administering after a first period of time, e.g., such methods may incur weight loss for three or four days or more after administration of a single dose. For example, a patient, after receiving a first dose and/or after receiving a subsequent dose, may continue to lose weight for three or four days or more without further administration of a disclosed composition. In some embodiments, administration of an initial first dose, or administration of a first and second dose (e.g., both administered in the same week), may provide about 0.3 kg to about 2 kg or more (e.g., about 0.5 kg to about 2 kg or more) of weight loss. Subsequent administration may result in further weight loss, until a target patient weight is achieved.

In some embodiments, the compositions described herein (e.g., a suspension lyophile composition) are administered, reconstituted, at a frequency of about every other day (e.g., every two days); one or two times a week; one, two or three times a week; two or three times a week; twice weekly (e.g. every 3 days, every 4 days, every 5 days, every 6 days or e.g. administered with an interval of about 2 to about 3 days between doses); every three to four days; once a week; every other week; twice monthly; once a month or even less often. It may be appreciated that methods that include administering a single dose on a less frequent basis, may, in some embodiments, be a method directed to amelioriating a condition. Alternatively, a composition (e.g., a reconstituted suspension lyophile composition) may be administered for a first period of time, withheld for a second period of time, and again optionally administered for a third period of time, e.g., alternate dosing regimens. For example, for the first period of time a patient may be administered a disclosed composition daily, every other day, every three, four or five days, biweekly, monthly, or yearly; during the second period of time (e.g. 1 day, 1 week, 2 weeks, 1 month) no composition is administered; and during e.g. a third period of time, the patient may be administered on a regimen similar or different to the first period of time, for example, every other day, every three, four or five days, biweekly, monthly, or yearly.

### Methods of Preparing Compositions

In some embodiments, methods of preparing a compositions described herein include (i) preparing sterile, particle size controlled ZGN-440; (ii) preparing a suspension of said material; (iii) filling a container (e.g., a vial or delivery device) with said material; and (iv) lyophilizing said material to provide a lyophile as described herein. It will be appreciated that step (i) is typically conducted under aseptic conditions so as to provide sterile drug substance.

The following exemplary processes can be used to prepare an appropriate particle size of ZGN-440 (i) microfluidization; (ii) jet milling; (iii) ball milling; and/or suspension homogenization. In some embodiments, there is no observable change in the polymorphic form when ZGN-440 Form A is either suspended in different aqueous diluents (even for extended times) or when ZGN-440 jet milled or microfluidized to reduce the particle size. In some embodiments, to maintain optimal suspension viscosity, it is preferable to perform microfluidization in the presence of poloxamer 188 alone (or optionally with a buffer) and introduce other excipients at a later point in the preparation of the compositions (see, e.g., Example 2). In some embodiments, jet milling of neat dry ZGN-440 and microfluidization of ZGN-440 in a poloxamer-containing suspension can provide ZGN-440 with a relatively small particle size ZGN-440 (e.g., D90 of less than 30 μm). See, e.g., Example 1.

In some embodiments, lyophilization can be performed at a pressure of about 200 mTorr. In some embodiments, lyophilization can be performed over a temperature range of from about −40° C. to about 25° C. In some embodiments, lyophiles have a shelf stability of at least 3 months (e.g., at least 6 months, at least 12 months, at least 18 months, at least 24 months, at least 30 months) at about 5° C.

In some embodiments, compositions described herein (e.g., lyophiles) are reconstituted with from 0.5 mL to about 1.0 mL (e.g., 0.71 mL) of a diluent (e.g., WFI and optionally Polysorbate 80). In certain embodiments, reconstitution is performed by manually rolling the container (e.g., vial, e.g., for about 1 minute) with minimal vial hold and syringe hold times (e.g., 10 and 15 minutes, respectively). In some embodiments, sonication (e.g., for 2 minutes can facilitate reconstitution.

### Kits

In some embodiments, kits are provided (e.g., ready to use kits or reconstitution kits), comprising a suspension lyophile composition and optionally one or more diluents (e.g., reconstitution vehicles, e.g., WFI). For example, the suspension lyophile composition and diluent if present may are provided (together or separately) in one or more containers (e.g., vials, such as reconstitution vials or unit dose vials; syringes, such as a pre-loaded syringe or a pre-loaded dual chamber syringe.

In certain embodiments, kits are provided that include a first container (e.g., vial) that includes a disclosed lyophile composition described herein and a second vial that contains a diluent for reconstitution (e.g., a diluent that includes WFI and Polysorbate 80). Such kits can further include administration/delivery materials, e.g., syringes and vial adaptors, such as two 1-ml syringes and two 510K approved and CE marked vial adapters per dose (e.g., as provided by West Pharmaceutical Services). The vial adapters can serve to simplify reconstitution of the lyophile, reduce the number of needles the patient or caregiver is exposed to and facilitate the dose preparation.

In certain embodiments, kits are provided that include a first container, such as a 2 mL vial, that contains a disclosed lyophile composition described herein. Such kits can further include a prefilled syringe, such as a 1 mL prefilled syringe, that contains a diluent for reconstitution (e.g., a diluent that includes WFI and Polysorbate 80), a Mixject™ vial adaptor, and a plunger rod.

In other embodiments, kits are provided that include a dual chamber cartridge, in which one of the chambers contains a disclosed lyophile composition described herein and the other chamber optionally includes a diluent. Examples of devices that incorporate a dual chamber cartridge include an auto-injector with integrated needle and automated diluent transfer, auto-injectors with user attached needle and manual diluent transfer, and pen injectors with user attached needle and manual diluent transfer.

In other embodiments, kits are provided that include a dual chamber syringe, for example, in which one of the chambers contains a disclosed lyophile composition described herein and the other chamber includes a diluent. With dual chamber syringe systems, generally the user meters the volume of drug delivered, as opposed to the device controlling the delivered volume as in systems based on dual chamber cartridges.

In other embodiments, kits are provided that include a device based on a diluent syringe and drug vial, for example, an auto-injector with staked needle on the diluent syringe, or a manual injector which houses a prefilled diluent syringe in the upper portion of the device and a vial containing a disclosed lyophile composition described herein in the lower portion.

In other embodiments, kits are provided that contain a device based on a drug syringe and diluent syringe, wherein a disclosed lyophile composition described herein and a diluent can both be filled into syringes for some device configurations. For example, the syringes can be housed in the device independently. In another example, the syringes can be nested concentrically.

In still other embodiments, the compositions and diluents described herein can be provided in separate kits, each being contained as described above.

### EXAMPLES

The invention now being generally described, it will be more readily understood by reference to the following examples which are included merely for purposes of illustration of certain aspects and embodiments of the present invention, and are not intended to limit the invention in any way.

### Example 1—ZGN Particle Size

The following processes may be used to prepare an appropriate particle size of ZGN-440 (i) microfluidization; (ii) jet milling; (iii) ball milling; and suspension homogenization. There is no change in the polymorphic form when ZGN-440 is either suspended in different aqueous diluents (e.g., for extended times) or when ZGN-440 jet milled or microfluidized to reduce the particle size.

A. Microfluidization

The microfluidization process was designed to affect particle size reduction to a concentrated suspension of drug substance (13.2 mg/g) in water for injection, with the addition of a surfactant (poloxamer 188, 0.36%) to maintain dispersion of the drug substance during microfluidization. The microfluidization step involves multiple passes through the microfluidizer's interaction chambers (via closed system recirculation) until the target particle size distribution is achieved. Subsequently, the concentrated suspension is diluted with additional sterile filtered poloxamer solution (based on in-process assay and batch weight) to a target concentration of 9 mg/g. This suspension is then further diluted with a sterile-filtered solution of the remaining excipients (povidone K12, trehalose, sodium phosphate salts) to a target suspension concentration of 6 mg/g (particle size reduction can be adversely affected by the presence of multiple excipients). The initial dilution of the suspension (based on IPC) with poloxamer solution allows compensation for drug substance losses that occur during microfluidization, and the use of a fixed ratio of suspension:excipient solution during the final dilution step. This process ensures a consistent composition of the final fill suspension. A final IPC assay test after dilution to 6 mg/mL allows for final minor adjustments to a fill weight as might be needed.

Microfluidization Process Parameters

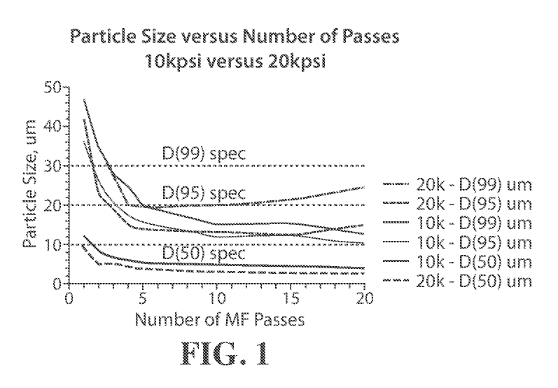

Microfluidization process parameters contributing to particle size reduction include operating pressure, channel diameters of interaction chambers and the number of passes of the suspension through the interaction chambers. Operating pressures of 10,000 psi and 20,000 psi were selected for characterization in terms of particle size distribution as a function of number of passes through the interaction chambers. Relatively consistent particle size profiles were observed with either operating pressure, with both demonstrating a particle size plateau by the 10^{th} pass using a 200 um interaction chamber in series with an 87 um chamber. (Other chambers may be used e.g., a 100 um chamber). There was evidence of slight increase in particle size beyond the 10^{th} pass with the higher operating pressure, which could be related to particle aggregation subsequent to processing, and the target pressure of 10,000 psi was then used for microfluidization. FIG. 1 illustrates the effects of operating pressure on particle size reduction as a function of number of passes through the microfluidizer.

Two different interaction chamber materials are available for the microfluidizer, ceramic and diamond. A study was conducted to evaluate whether either material had any advantages in terms of particle size reduction efficiency. No apparent differences were observed in the particle size reduction profiles. Ceramic interaction chambers were selected for the GMP microfluidizer.

A potential failure mode, plugging (requiring disassembly and clean-out of the chamber, an operation that must be avoided in a GMP manufacturing environment) may be mitigated by processing the suspension initially through only the larger 200 micron chamber for several passes, followed by continuing passes through both chambers in series. This was accomplished by reconfiguring the microfluidizer with a diverter valve, allowing the 87 micron chamber to be bypassed for initial passes while maintaining a closed system. It was found that three initial passes through the 200 micron chamber is sufficient to deagglomerate the drug substance and prevent plugging on subsequent passage through the 87 micron chamber.

Agitation in Feed Vessel and Collection Vessel

The suspension is continuously stirred in both the microfluidizer feed vessel and collection vessel, with inclusion of a recirculation loop in the collection reservoir to further mix the suspension in the collection reservoir. Agitation conditions are controlled in the feed vessel (inlet to the microfluidizer) and the collection vessel at the microfluidizer outlet to avoid turbulent mixing and therefore minimize agglomeration and subsequent drug substance losses, while maintaining homogeneity of the suspended drug substance crystals. Additionally, increasing poloxamer 188 concentration reduced API agglomeration and losses during microfluidization.

The microfluidization process selected for the ZGN-440 for injectable suspension formulation involved the following sequence, performed via closed system recirculation: 3 passes through isolated 200 micron chamber at 10,000 psi; and 10 passes through 200 micron and 87 micron chambers connected in series at 10,000 psi. In some cases, an additional high pressure over-processing step can be employed (e.g., 5 passes at 20,000 psi). In other cases, lower pressures can be used (3 passes through isolated 200 micron chamber at 5,000 psi; and 10 passes through 200 micron and 87 micron chambers connected in series at 5,000 psi, followed by 7 passes at 10,000 psi. Some foaming can be observed under these conditions. The processing steps above can be sensitive to pH changes, and a drop-off in performance has been observed when operating below pH 8.1. Finally, crystals having a relative high aspect ratio (e.g., 1:25-1:50) and thickness (e.g., 5-10 micron) present a greater risk of plugging the system.

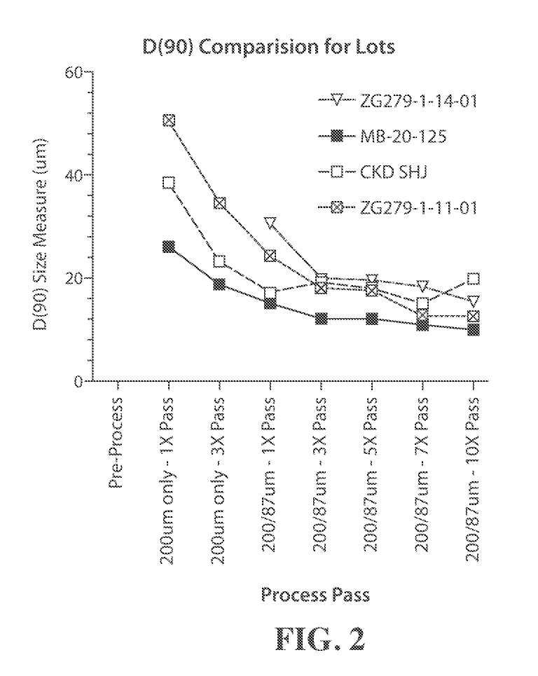

This selected microfluidization process was applied to several different drug substance lots. As illustrated in FIG. 2, a reasonably consistent particle size reduction plateau is achieved, despite significant differences in particle size of the in-going drug substance lots.

B. Jet Milling

Jet milling was conducted with ZGN-440 having an initial D90 of 211.9 μm prior to jet milling. Table 1 shows the particle size under several different jet milling conditions. The XRPD spectra of a NAT jet milled sample of ZGN-440 demonstrated that the material retained its crystallinity and polymorphic form after jet milling. Similarly, sample FPS5 was shown to be crystalline and the desired polymorph after jet milling and to have the same XRPD, TGA and DSC as other samples of ZGN-440.

TABLE 1

| Experiment | Air Pressure | Feed Rate | D10 (mm) | D50 (mm) | D90 (mm) | Scale |
|---|---|---|---|---|---|---|
| NAT | 30 psi (2 bar) | 0.36 g/min | 1.7 | 9.4 | 25.1 | 2 g |
| FPS/1 | 7 bar | 0.5 g/min | 1.1 | 2.4 | 5.2 | 2.2 g |
| FPS/2 | 5 bar | 0.5 g/min | 1.1 | 3.0 | 6.9 | 1.9 g |
| FPS/3 | 3 bar | 1 g/min | 1.2 | 3.9 | 9.5 | 1.9 g |
| FPS/4 | 1 bar | 4 g/min | 1.5 | 6.5 | 13.2 | 1.95 g |
| FPS/5 | 1 bar | 4 g/min | 1.2 | 6.1 | 14.3 | 24 g |

For FPS5, jet milled ZGN-440 was recovered in greater than 90% yield. Table 1 demonstrates that ZGN-440 can be jet milled with control over the resulting particle size, and that ZGN-440 retains its crystallinity and its polymorphic structure. The particle size generated is suitable for use with a 29-gauge to 31-gauge needle.

### Example 2—Process Description for ZGN-440 for Injectable Suspension

1. Prepare 0.36% w/w poloxamer solution in WFI and mix for ≧5 minutes until solution is clear.

2. Sample poloxamer solution for pre-sterilization bioburden.

3. Pass the poloxamer solution through two redundant 0.2 micron sterile filters (Sartorius Stedim Sartobran P 0.2 μm polyethersulfone filters with integral 0.45 μm prefilter) connected in series into a pre-sterilized feed vessel equipped with an agitator.

4. Initiate mixing of poloxamer solution in the feed vessel at 200 rpm±100 rpm.

5. Aseptically connect the sterile ZGN-440 drug substance Hyclone bag to the Poloxamer-containing feed vessel and slowly add the ZGN-440 to the poloxamer solution over approximately 10 to 20 minutes while maintaining agitation at 200 rpm±100 rpm.

6. Agitate at 200 rpm±100 rpm until visually homogeneous.

7. Prime the pre-sterilized microfluidizer with sterile filtered 0.36% w/w poloxamer solution.

8. Configure the microfluidizer so that the 87 micron interaction chamber is bypassed, by opening and closing appropriate integrated bypass valves.

9. Set the water bath temperature (attached to the heat exchangers at the microfluidizer outlet) to a set point of 2° C.

10. Pass the ZGN-440 suspension from Step 6 through the microfluidizer at an operating pressure of 5,000 to 10,000 psi, and begin collection in a pre-sterilized receiving vessel.

11. Begin agitation in the Collection Vessel at 60 rpm when the volume reaches approximately 1 L. When the volume reaches approximately 2 L, increase agitation to 150-250 rpm. Reduce agitation if foaming occurs.

12. When the feed vessel is ˜95% depleted, initiate recirculation of the contents of the receiving vessel back into the feed vessel, until the receiving vessel is depleted ˜95%.

13. Repeat Steps 9 through 11 for a total of 3 passes.

14. Configure the microfluidizer to include both the 200 micron and 87 micron interaction chambers connected in series.

15. Pass the suspension from Step 12 through the microfluidizer at 10,000 psi for a total of 10 passes, with recirculation after each pass when the feed vessel reaches ˜95% depletion. The flow rate during microfluidization through both chambers, at an operating pressure of 10,000 psi, is approximately 244 mL/minute.

16. Record weight of final microfluidized suspension.

17. Sample the suspension for PSD (information only), pH (information only) and assay (IPC-1).

18. Based on IPC-1 assay and batch weight from Step 16, dilute the suspension to 9.0 mg/g with the filtered 0.36% w/w poloxamer solution.

19. Record suspension weight after dilution.

20. Prepare excipient solution (trehalose, povidone, monobasic sodium phosphate, dibasic sodium phosphate, WFI), and mix for ≧5 minutes until a clear solution is achieved.

21. Pass the excipient solution through two redundant 0.2 micron sterile filters (Sartorius Stedim Sartobran P 0.2 μm polyethersulfone filters with integral 0.45 nm prefilter) connected in series into a pre-sterilized container.

22. Based on suspension weight from Step 19, aseptically add a sufficient quantity of the sterile-filtered excipient solution to achieve a target ZGN-440 concentration of 6.0 mg/g.

23. Sample the diluted suspension for PSD (information only), pH (information only) and final suspension assay (IPC-2).

24. The ZGN-440 suspension should be continuously agitated at ambient conditions (50 to 100 rpms), protected from light, while awaiting IPC-2 result.

25. Transfer the suspension to the Grade A filling suite, initiate recirculation in the fill vessel target 75 rpms), and continue agitation (50 to 100 rpms).

26. Perform environmental monitoring for viable and non-viable particulates per SOP during operation in the Grade A area.

27. Based on IPC-2 result, calculate Target Fill Weight (TFW) to achieve 3.00 mg ZGN-440 per vial. (Nominal TFW is 0.50 g per vial for a 3.00 mg/g suspension concentration.)

28. Fill vials at calculated target fill weight. Maintain agitation and recirculation during filling.

29. Check fill weights periodically (approximately every 150 to 250 vials, or 2 to 3 vials per lyo tray) during filling and adjust filler settings if necessary to maintain fill weight. Fill weight Alert Limits: TFW±0.03 g. Fill weight Action Limits: TFW±0.05 g.

30. Partially insert stoppers in lyophilization position and load vials onto lyophilizer trays in the Grade A area.

31. Upon complete packing of each lyophilizer tray, load the trays onto presterilized depyrogenated lyophilizer shelves pre-cooled to −40° C.

32. Hold vials in the lyophilizer at −40° C. for NLT 8 hours.

33. Evacuate chamber to maintain a pressure of 200 microns.

34. Ramp the shelf temperature to −25° C. at a rate of 0.1° C. per minute.

35. Hold shelf temperature at −25° C. for NLT 80 hours.

36. Ramp the shelf temperature to 25° C. at a rate of 0.1° C. per minute.

37. Hold shelf temperature at 25° C. for NLT 8 hours.

38. Back-fill freeze dryer with sterile-filtered nitrogen to ambient pressure and seat stoppers.

39. Remove vials from the lyophilizer shelves, into the Grade A filling/capping suite.

40. Apply overseals.

41. Perform 100% visual inspection.

### Example 3—Lyophile Formulation

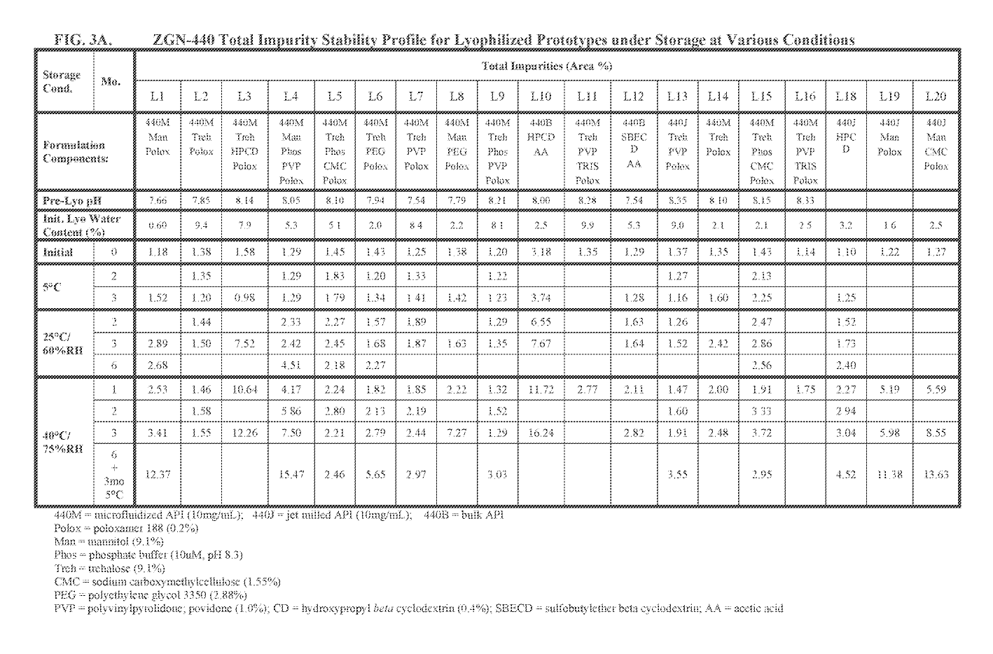

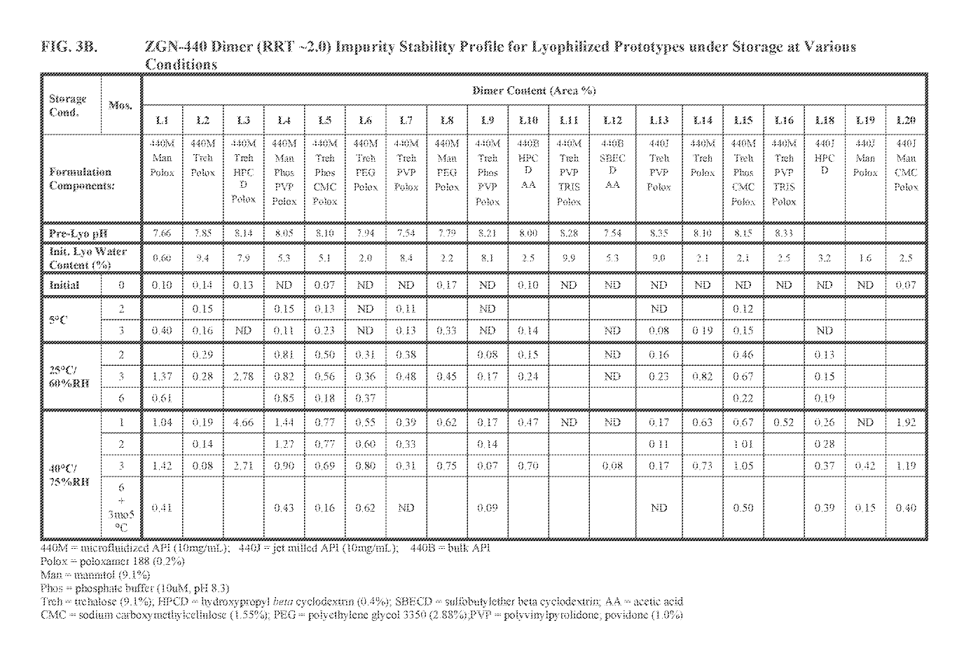

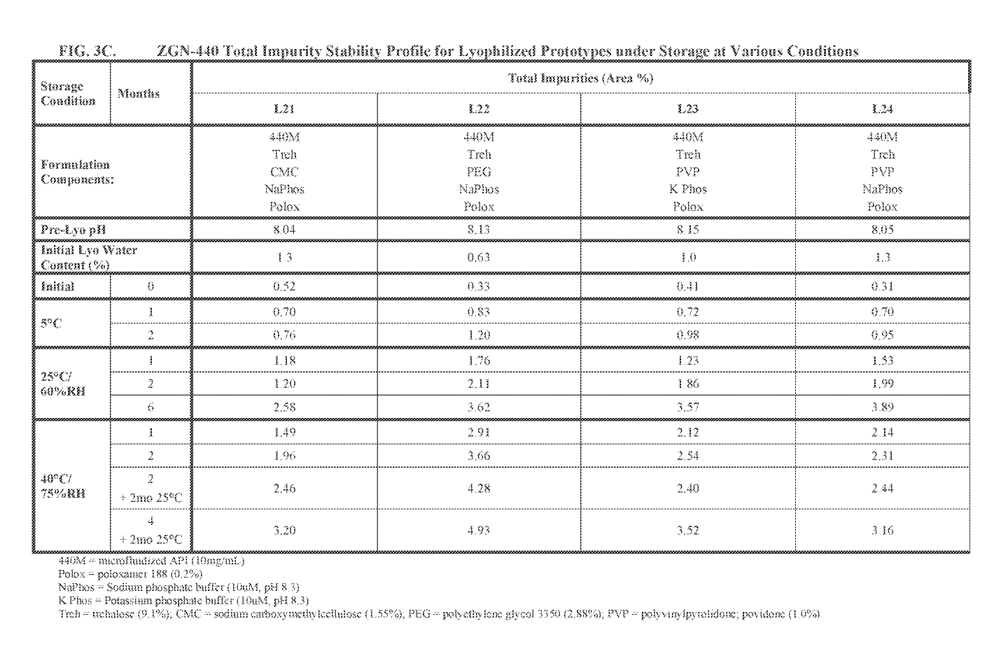

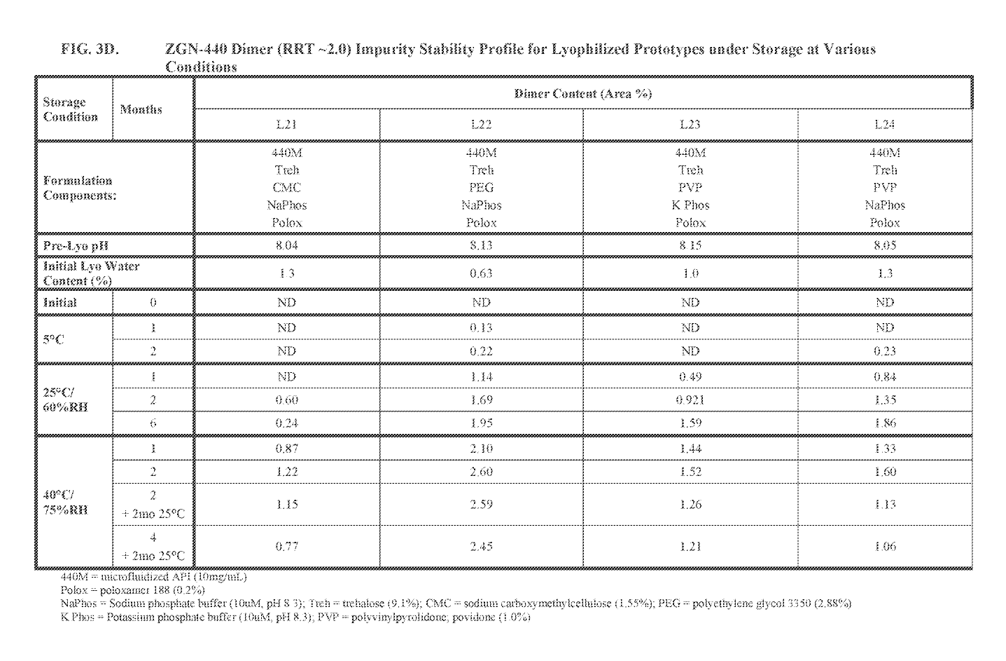

Various compositions were prepared to evaluate storage stability under a variety of conditions. Stability results are provided in FIGS. 3A, 3B, 3C, and 3D. As indicated in these tables, while mannitol is the most commonly used bulking agent for lyophile formulations, it was surprisingly found that lyophile stability in mannitol-containing formulations was significantly inferior to stability of formulations containing trehalose as a bulking agent.

Further studies examined three viscosity agents: NaCMC, PEG 3350, and PVP. Lyophiles with PVP showed improved stability in terms of formation of the dimer degradant compared with lyophiles containing PEG 3350, as shown in the tables, while lyophiles containing NaCMC appeared to show inferior reconstitution properties relative to PVP-containing lyophiles.

Exemplary compositions are provided in Tables 2-5.

TABLE 2
Composition of the Drug Product: 2.4 mg Dose (3 mg per vial)

| | | | In-Use | |
|---|---|---|---|---|
| | | Unit | Concentration | |
| | Unit | Concentration | (mg/mL upon | |
| | Composition | (% w/w in | reconstitution) | |
| Ingredient | (mg per vial) | lyophile) | (1) | Function |

Drug Substance

| | | | | |
|---|---|---|---|---|
| ZGN-440 | 3.00 | 4.84 | 4.00 | Active |
| | | | | Ingredient |

Excipients

| | | | | |
|---|---|---|---|---|
| Poloxamer 188 | 1.20 | 1.93 | 1.60 | Dispersing aid |
| Trehalose dihydrate | 51.93 | 83.71 | 69.24 | Bulking agent/ |
| | | | | Tonicity |
| | | | | modifier |
| Povidone K17 | 2.88 | 4.64 | 3.84 | Suspending |
| | | | | agent |
| Dibasic sodium | 2.95 | 4.76 | 3.94 | Buffer |
| phosphate, | | | | |
| heptahydrate | | | | |
| Monobasic sodium | 0.072 | 0.12 | 0.096 | Buffer |
| phosphate, | | | | |
| monohydrate | | | | |

Processing Aids

| | | | | |
|---|---|---|---|---|
| Sterile water for | (2) | (2) | (2) | Processing aid |
| injection | | | | |
| Nitrogen | (3) | (3) | (3) | Processing aid |

Contribution from Reconstitution Diluent

| | | | | |
|---|---|---|---|---|
| Sterile water for | N/A | N/A | 939.55 | Vehicle |
| injection | | | | |
| Polysorbate 80 | N/A | N/A | 4.74 | Dispersing aid |
| Total | 62.04 (4) | 100.00 | 1027.00 (5) | |

(1) Reconstitution with 0.71 mL of ZGN-440 Sterile Diluent produces 0.75 mL of suspension with a ZGN-440 concentration of 4 mg/mL and a pH of approximately 8.3.
(2) Removed during lyophilization
(3) Used to backfill freeze dryer and overlay vials at the completion of the lyophilization cycle
(4) Reflects total weight on an anhydrous basis
(5) Based on a reconstituted suspension density of 1.027 g/mL.

TABLE 3
Composition of the Drug Product: 1.8 mg Dose (2.25 mg per vial)

| | | | In-Use | |
|---|---|---|---|---|
| | | Unit | Concentration | |
| | Unit | Concentration | (mg/mL upon | |
| | Composition | (% w/w in | reconstitution) | |
| Ingredient | (mg per vial) | lyophile) | (1) | Function |

Drug Substance

| | | | | |
|---|---|---|---|---|
| ZGN-440 | 2.20 | 3.67 | 3.00 | Active |
| | | | | Ingredient |

Excipients

| | | | | |
|---|---|---|---|---|
| Poloxamer 188 | 1.20 | 1.96 | 1.60 | Dispersing aid |
| Trehalose dihydrate | 51.93 | 84.74 | 69.24 | Bulking agent/ |
| | | | | Tonicity |
| | | | | modifier |
| Povidone K17 | 2.88 | 4.70 | 3.84 | Suspending |
| | | | | agent |
| Dibasic sodium | 2.95 | 4.82 | 3.94 | Buffer |
| phosphate, | | | | |
| heptahydrate | | | | |
| Monobasic sodium | 0.072 | 0.12 | 0.096 | Buffer |
| phosphate, | | | | |
| monohydrate | | | | |

Processing Aids

| | | | | |
|---|---|---|---|---|
| Sterile water for | (2) | (2) | (2) | Processing aid |
| injection | | | | |
| Nitrogen | (3) | (3) | (3) | Processing aid |

Contribution from Reconstitution Diluent

| | | | | |
|---|---|---|---|---|
| Sterile water for | N/A | N/A | 940.55 | Vehicle |
| injection | | | | |
| Polysorbate 80 | N/A | N/A | 4.74 | Dispersing aid |
| Total | 61.29 (4) | 100.00 | 1027.00 (5) | |

(1) Reconstitution with 0.71 mL of ZGN-440 Sterile Diluent produces 0.75 mL of suspension with a ZGN-440 concentration of 3 mg/mL and a pH of approximately 8.3.
(2) Removed during lyophilization
(3) Used to backfill freeze dryer and overlay vials at the completion of the lyophilization cycle
(4) Reflects total weight on an anhydrous basis
(5) Based on a reconstituted suspension density of 1.027 g/mL.

TABLE 4
Composition of the Drug Product: 1.2 mg Dose (1.5 mg per vial)

| | | | In-Use | |
|---|---|---|---|---|
| | | Unit | Concentration | |
| | Unit | Concentration | (mg/mL upon | |
| | Composition | (% w/w in | reconstitution) | |
| Ingredient | (mg per vial) | lyophile) | (1) | Function |

Drug Substance

| | | | | |
|---|---|---|---|---|
| ZGN-440 | 1.50 | 2.48 | 2.00 | Active |
| | | | | Ingredient |

Excipients

| | | | | |
|---|---|---|---|---|
| Poloxamer 188 | 1.20 | 1.98 | 1.60 | Dispersing aid |
| Trehalose dihydrate | 51.93 | 85.79 | 69.24 | Bulking agent/ |
| | | | | Tonicity |
| | | | | modifier |
| Povidone K17 | 2.88 | 4.76 | 3.84 | Suspending |
| | | | | agent |
| Dibasic sodium | 2.95 | 4.88 | 3.94 | Buffer |
| phosphate, | | | | |
| heptahydrate | | | | |
| Monobasic sodium | 0.072 | 0.12 | 0.096 | Buffer |
| phosphate, | | | | |
| monohydrate | | | | |

Processing Aids

| | | | | |
|---|---|---|---|---|
| Sterile water for | (2) | (2) | (2) | Processing aid |
| injection | | | | |
| Nitrogen | (3) | (3) | (3) | Processing aid |

Contribution from Reconstitution Diluent

| | | | | |
|---|---|---|---|---|
| Sterile water for | N/A | N/A | 941.55 | Vehicle |
| injection | | | | |
| Polysorbate 80 | N/A | N/A | 4.74 | Dispersing aid |
| Total | 60.54 (4) | 100.00 | 1027.00 (5) | |

(1) Reconstitution with 0.71 mL of ZGN-440 Sterile Diluent produces 0.75 mL of suspension with a ZGN-440 concentration of 2 mg/mL and a pH of approximately 8.3.
(2) Removed during lyophilization
(3) Used to backfill freeze dryer and overlay vials at the completion of the lyophilization cycle
(4) Reflects total weight on an anhydrous basis
(5) Based on a reconstituted suspension density of 1.027 g/mL.

TABLE 5
Composition of the Drug Product: 0.6 mg Dose (0.75 mg per vial)

| | | | In-Use | |
|---|---|---|---|---|
| | | Unit | Concentration | |
| | Unit | Concentration | (mg/mL upon | |
| | Composition | (% w/w in | reconstitution) | |
| Ingredient | (mg per vial) | lyophile) | (1) | Function |

Drug Substance

| | | | | |
|---|---|---|---|---|
| ZGN-440 | 0.75 | 1.25 | 1.00 | Active |
| | | | | Ingredient |

Excipients

| | | | | |
|---|---|---|---|---|
| Poloxamer 188 | 1.20 | 2.01 | 1.60 | Dispersing aid |
| Trehalose dihydrate | 51.93 | 86.86 | 69.24 | Bulking agent/ |
| | | | | Tonicity |
| | | | | modifier |
| Povidone K17 | 2.88 | 4.82 | 3.84 | Suspending |
| | | | | agent |
| Dibasic sodium | 2.95 | 4.94 | 3.94 | Buffer |
| phosphate, | | | | |
| heptahydrate | | | | |
| Monobasic sodium | 0.072 | 0.12 | 0.096 | Buffer |
| phosphate, | | | | |
| monohydrate | | | | |

Processing Aids

| | | | | |
|---|---|---|---|---|
| Sterile water for | (2) | (2) | (2) | Processing aid |
| injection | | | | |
| Nitrogen | (3) | (3) | (3) | Processing aid |

Contribution from Reconstitution Diluent

| | | | | |
|---|---|---|---|---|
| Sterile water for | N/A | N/A | 942.55 | Vehicle |
| injection | | | | |
| Polysorbate 80 | N/A | N/A | 4.74 | Dispersing aid |
| Total | 62.04 (4) | 100.00 | 1027.00 (5) | |

(1) Reconstitution with 0.71 mL of ZGN-440 Sterile Diluent produces 0.75 mL of suspension with a ZGN-440 concentration of 1 mg/mL and a pH of approximately 8.3.
(2) Removed during lyophilization
(3) Used to backfill freeze dryer and overlay vials at the completion of the lyophilization cycle
(4) Reflects total weight on an anhydrous basis
(5) Based on a reconstituted suspension density of 1.027 g/mL.

### Example 4. Evaluation of Lyophilized Product Delivery

Reconstitution of lyophilized cakes containing Tween80 (Polysorbate 80) with water for injection were compared to reconstituting lyophilized cakes without Tween 80. No delivery issues were found when Tween 80 was present in the lyophile (however, such lyophiles can be friable resulting in powdery material in the vial). In addition, although jet-milled material does not appear as aesthetic as the microfluidized product (crystals coating wall are visible), it did appear to provide more consistent delivery. Even with no vial hold and no syringe hold, control microfluidized product is showing 90% recovery. Jet milled shows visual crystals on vial walls, but loses only 3-4% ZGN-440. See Table 6. A homogeneous suspension upon reconstitution of the ZGN-440 lyophile was to enable accurate dose withdrawal and delivery. Among a series of nine aqueous vehicle compositions examined (WFI, 1.6% NaCMC, pH 7.5, 0.5% Tween 80, 0.5% Poloxamer, 5% ethanol, 5% propylene glycol, 5% PEG3350, 5% PEG400), only the Tween 80 solution produced a complete and homogeneous suspension upon reconstitution of the lyophile. Other diluents resulted in varying degrees of flocculated API which adhered to the vial walls, which would not be accessible upon dose withdrawal

TABLE 6

| | | | HPLC | |
|---|---|---|---|---|
| | | | Delivery, | |
| Sample | Description | Diluent | % label | Observations |
| 1 | Microfluidized ZGN-440 | WFI | 94.1 | No delivery issues; intact |
| | lyophilized with Tween 80 | | (2.1%) | lyophilized cake |
| | (A) | | | |
| 2 | Microfluidized ZGN-440 | 0.5% | 89.9 | No delivery issues; intact |
| | lyophilized without Tween | Tween 80 | (0.4%) | lyophilized cake |
| | 80 (A) | | | |
| 3 | Microfluidized ZGN-440 | WFI | 88.7 | No delivery issues; intact |
| | lyophilized with Tween 80 | | (0.3%) | lyophilized cake |
| | (B) | | | |
| 4 | Microfluidized ZGN-440 | 0.5% | 91.5 | No delivery issues; intact |
| | lyophilized without Tween | Tween 80 | (0.2%) | lyophilized cake |
| | 80 (A) | | | |
| 5 | Jet milled ZGN-440 with | WFI | 97.3% | No delivery issues; intact |
| | Tween 80 | | (2.2%) | lyophilized cake. Particles stuck |
| | | | | to the via walls after hand roll |
| 6 | Jet milled ZGN-440 | 0.5% | 96.3 | No delivery issues; crumbled |
| | without Tween 80 | Tween 80 | (4.1%) | lyophilized cake. Particles and |
| | | | | small clumps stuck to the vial |
| | | | | walls after hand roll. Small |
| | | | | clumps present in the syringe. |

Each sample run in triplicate; samples marked with (A) and (B) run by different investigator.
Sample: dose = 1.8 mg; needle = 27 G; delivery volume = 0.45 mL.
Sample preparation for delivery: 1 minute vial hand roll; vial and syringe hold time = 0.

### Example 5. Lyophile Formulation—Content Uniformity

The reconstituted formulation of the lyophiles in Example 3 exhibit relatively high ZGN-440 content uniformity in filled vials. A study was conducted using production equipment to evaluate the accuracy of vial filling and API content consistency across the duration of filling approximately 12,000 vials. Results indicated that vial to vial fill accuracy remained consistent throughout the fill of a full-scale batch. Although there was some downward trend in label claim by mg/vial, this reflected a downward trend in fill volume (rather than suspension concentration), since the assay per gram of suspension remained constant throughout the fill. The consistency of the mg/g values shown in FIG. 4 demonstrates that the suspension maintains excellent API homogeneity in the bulk suspension throughout the duration of a production-scale filling operation.

### Example 6. Reconstitution Vehicle

A homogeneous suspension is prepared upon reconstitution of the ZGN-440 lyophile as in Example 3. Nine aqueous vehicle compositions were examined (WFI, 1.6% NaCMC, pH 7.5, 0.5% Tween 80, 0.5% Poloxamer, 5% ethanol, 5% propylene glycol, 5% PEG3350, 5% PEG400). The Tween 80 solution produced a complete and homogeneous suspension upon reconstitution of the lyophile. Other diluents resulted in varying degrees of flocculated ZGN-440, which adhered to the vial walls, which would not be accessible upon dose withdrawal.

### Example 7. Delivery Failure Versus Particle Size

FIG. 5 is a graph summarizing delivery failure versus particle size for the delivery studies carried out using ZGN-440. Focusing on 2 minutes of sonication, the data shows that the best results were obtained when the ratio of D(90) to the needle internal diameter (ID) is less than 19%. For example, a 30 G needle with a 159 um ID would be suitable with a D(90) specification of <30.2 um. The use of sonication was shown to have a significant impact on acceptable delivery. See FIG. 6.

### Example 8: Pharmacokinetic Study in Rats

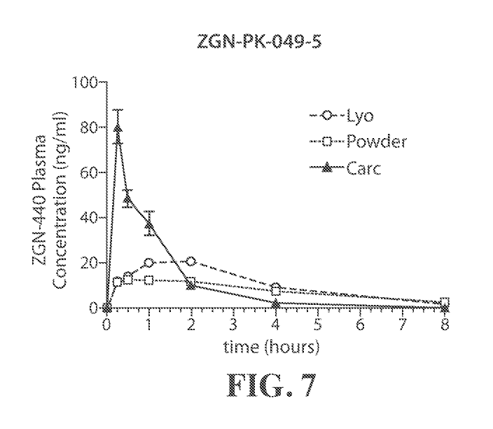

An evaluation of three product formulations (lyophilized as in Example 3, powder fill was conducted. Three groups of rats were used as below. No adverse response observed for SC injection for any group. The PK plots are shown in FIG. 7. No clinical observations post administration. See the tables below.

TABLE 7

| | | | | Dose, | Conc., | | |
|---|---|---|---|---|---|---|---|
| | | Number | | active | active | Dose | Dose |
| | | of | Admin. | basis | basis | Volume | Volume |
| Group | Description | Animals | Route | (mg/kg) | (mg/mL) | (mL/kg) | (mL) |
| 1 | ZGN-440 Lyo | 3M, 3F | SC | 1.0 | 4.0 | 0.25 | 0.100 |
| | Clinical Product | | | | | | |
| 2 | ZGN-440 Powder | 3M, 3F | SC | 1.0 | 4.0 | 0.25 | 0.100 |
| | Fill Clinical | | | | | | |
| | Product | | | | | | |
| 3 | ZGN-440 | 3M, 2F | SC | 1.0 | 3.0 | 0.33 | 0.133 |
| | Carcinogenicity | | | | | | |
| | Study Product | | | | | | |

TABLE 8

| Group | | | | Number | Dose | Collection |
|---|---|---|---|---|---|---|
| ID | Compound | Product Description | Route | Animals | (mg/kg) | Schedule |
| 1 | ZGN-440 | Reconstituted | SC | 3M, 3F | 1.0 | t = 0, 0.25, 0.5, |
| Lyo | | Lyophilized | | | | 1, 2, 4, 8, 12, |
| | | suspension | | | | and 24 hours |
| | | (equivalently | | | | after dosing on |
| | | reconstituted with | | | | Day 1 |
| | | 0.5% Tween to 4.0 mg/mL) | | | | |
| 2 | ZGN-440 | (8.3 mg clinical vial | SC | 3M, 3F | 1.0 | t = 0, 0.25, 0.5, |
| Powder | | reconstituted with | | | | 1, 2, 4, 8, 12, |
| Fill | | ZGN-440 Diluent to | | | | and 24 hours |
| | | 4.0) | | | | after dosing on |
| | | | | | | Day 1 |
| 3 | ZGN-440 | 3.0 mg/mL ZGN-440 | SC | 3M, 2F¹ | 1.0 | t = 0, 0.25, 0.5, |
| Carcin. | | solution in 100 mM | | | | 1, 2, 4, 8, 12, |
| | | phosphate buffer, pH | | | | and 24 hours |
| | | 6.0 | | | | after dosing on |
| | | | | | | Day 1 |

### Example 9: Pediatric Product Formulation

Overview

A study to prepare ZGN-440 pediatric products at 1.8, 1.2, 0.6 and 0.3 mg dose levels using microfluidization and lyophilization techniques was conducted. The product fill volume was 0.50 mL; the minimum due to fill tolerance considerations. Excipient levels remained constant across pediatric dose levels and matched the current clinical product. All prototypes were evaluated and placed on long term storage stability at 5° C., 25° C. and 40° C. for up to 12 months.

The materials used in the study included the ZGN-440 drug substance; excipients including trehalose dihydrate, disodium phosphate heptahydrate, monosodium phosphate monohydrate, poloxamer 188, povidone K17, and water for injection (WFI); and fill product components including vial (2 mL clear, Type 1 tubular borosilicate glass, 13 mm neck), stopper (13 mm V2-F451W FluroTec B2-TR, 4432/50 chlorobutyl), and seal (13 mm aluminum, flip-off Truedge, matte blue).

Table 9 gives a summary of the ZGN-440 pediatric fill product formulation 1-8 evaluated in the study. Table 9A gives a summary of the pediatric fill product formulations 9-12, without PVP and with Tween 80 in the lyophile.

TABLE 9
Summary of Pediatric Experimental Design Fill Product Formulations

| Reference Product Number | | | | | | | |
|---|---|---|---|---|---|---|---|
| [1] | [2] | [3] | [4] | [5] | [6] | [7] | [8] |
| Description | | | | | | | |
| Control | | Pediatric | | Pediatric without PVP | | | |
| Dose Level (mg/dose) | | | | | | | |
| 2.4 | 1.8 | 1.8 | 0.30 | 1.8 | 1.2 | 0.60 | 0.30 |

Fill Product Amounts (mg/vial)

| | | | | | | | | |
|---|---|---|---|---|---|---|---|---|
| Beloranib (ZGN-440) | 3.0 | 2.25 | 2.25 | 0.375 | 2.25 | 1.5 | 0.75 | 0.375 |
| Poloxamer | 1.2 | 1.2 | 1.2 | 1.2 | 1.2 | 1.2 | 1.2 | 1.2 |
| Trehalose dihydrate | 51.93 | 51.93 | 51.93 | 51.93 | 51.93 | 51.93 | 51.93 | 51.93 |
| PVP | 2.88 | 2.88 | 2.88 | 2.88 | 0 | 0 | 0 | 0 |
| Disodium Phos Heptahydrate | 2.95 | 2.95 | 2.95 | 2.95 | 2.95 | 2.95 | 2.95 | 2.95 |
| Monosodium Phos Monohydrate | 0.07 | 0.07 | 0.07 | 0.07 | 0.07 | 0.07 | 0.07 | 0.07 |
| Water | 708.220 | 708.970 | 708.970 | 710.845 | 711.850 | 712.600 | 713.350 | 713.725 |

TABLE 9A
Summary of Pediatric Experimental Design Fill
Product Formulations with Tween 80 in Lyophile

| Reference Product Number | [9] | [10] | [11] | [12] |
|---|---|---|---|---|
| Description | Pediatric without PVP with Tween 80 | | | |
| Dose Level (mg/dose) | 1.8 | 1.2 | 0.60 | 0.30 |

Fill Product Amounts (mg/vial)

| | | | | |
|---|---|---|---|---|
| Beloranib (ZGN-440) | 2.25 | 1.5 | 0.75 | 0.375 |
| Poloxamer | 1.2 | 1.2 | 1.2 | 1.2 |
| Trehalose dihydrate | 51.93 | 51.93 | 51.93 | 51.93 |
| PVP | 0 | 0 | 0 | 0 |
| Disodium Phos Heptahydrate | 2.95 | 2.95 | 2.95 | 2.95 |
| Monosodium Phos Monohydrate | 0.07 | 0.07 | 0.07 | 0.07 |
| Polysorbate 80 (Tween 80) | 3.55 | 3.55 | 3.55 | 3.55 |
| Water | 708.299 | 709.049 | 709.799 | 710.174 |

Manufacture of Product [1]

Excipient Sub-Mix

| Ingredient | Wt. % |
|---|---|
| Trehalose dihydrate | 31.16 |
| PVP K17 | 1.728 |
| Poloxamer 188 | 0 |
| Disodium Phos | 1.772 |
| Monosodium Phos | 0.043 |
| WFI | 65.297 |

2.4 mg/dose product [1]

| Ingredient | Wt. % |
|---|---|
| ZGN-440 | 0.600 |
| Poloxamer 188 | 0.240 |
| Trehalose Dihydrate | 10.387 |
| Povidone K17 | 0.576 |
| Disodium Phos | 0.591 |
| Monosodium Phos | 0.014 |
| WFI | 87.592 |

Microfluidization was performed on 13.2 mg/ml of ZGN-440 in a 0.36% Poloxamer 188 microfluidization (MF) vehicle. The suspension was then diluted with additional portions of the MF vehicle to a concentration of 9.0 mg/ml of ZGN-440. The resulting suspension (⅔ by weight) was combined with an excipient sub-mix (⅓ by weight, see table above) to give a 6 mg/ml fill product. The resulting fill product (0.50 ml) was added to a vial, lyophilized, and sealed.

Manufacture of Product [2]

Excipient Sub-Mix

| Ingredient | Wt. % |
|---|---|
| Trehalose dihydrate | 31.16 |
| PVP K17 | 1.728 |
| Poloxamer 188 | 0 |
| Disodium Phos | 0 |
| Monosodium Phos | 0 |
| Water | 67.112 |

1.8 mg/dose product [2]

| Ingredient | Wt. % |
|---|---|
| ZGN-440 | 0.450 |
| Poloxamer 188 | 0.240 |
| Trehalose Dihydrate | 10.387 |
| Disodium Phos | 0.591 |
| Monosodium Phos | 0.014 |
| Water | 88.318 |

Microfluidization was performed on 13.2 mg/ml of ZGN-440 in a microfluidization (MF) vehicle containing 0.36% Poloxamer 188 in 30 mM phosphate buffer at pH 8.3. The suspension was then diluted with additional portions of the MF vehicle to a concentration of 6.75 mg/ml of ZGN-440. The resulting suspension (⅔ by weight) was combined with an excipient sub-mix (⅓ by weight, see table above) to give a 4.5 mg/ml fill product. The resulting fill product (0.50 ml) was added to a vial, lyophilized, and sealed.

Manufacture of Products [3] and [4]

Excipient Sub-Mix

| Ingredient | Wt. % |
|---|---|
| Trehalose dihydrate | 20.773 |
| PVP K17 | 1.152 |
| Poloxamer 188 | 0.12 |
| Disodium Phos | 0 |
| Monosodium Phos | 0 |
| Water | 77.955 |

1.8 mg/dose product [3]

| Ingredient | Wt. % |
|---|---|
| ZGN-440 | 0.450 |
| Poloxamer 188 | 0.240 |
| Trehalose Dihydrate | 10.387 |
| Povidone K17 | 0.576 |
| Disodium Phos | 0.591 |
| Monosodium Phos | 0.014 |
| Water | 87.742 |

Blank fill product

| Ingredient | Wt. % |
|---|---|
| ZGN-440 | 0 |
| Poloxamer 188 | 0.240 |
| Trehalose Dihydrate | 10.387 |
| Povidone K17 | 0.576 |
| Disodium Phos | 0.591 |
| Monosodium Phos | 0.014 |
| Water | 88.192 |

0.3 mg/dose product [4]

| Ingredient | Wt. % |
|---|---|
| ZGN-440 | 0.075 |
| Poloxamer 188 | 0.240 |
| Trehalose Dihydrate | 10.387 |
| Povidone K17 | 0.576 |
| Disodium Phos | 0.591 |
| Monosodium Phos | 0.014 |
| Water | 88.117 |

Microfluidization was performed on 13.2 mg/ml of ZGN-440 in a microfluidization (MF) vehicle containing 0.36% Poloxamer 188 in 40 mM phosphate buffer at pH 8.3. The suspension was then diluted with additional portions of the MF vehicle to a concentration of 9.0 mg/ml of ZGN-440. The resulting suspension (½ by weight) was combined with an excipient sub-mix (½ by weight, see table above) to give a 4.5 mg/ml fill product. The resulting fill product (0.50 ml) was added to a vial, lyophilized, and sealed to give [3].

The 4.5 mg/ml fill product (⅙ by weight) was combined with a blank fill product (⅚ by weight, see table above) to give a 0.75 mg/ml fill product. This material (0.5 ml) was added to a vial, lyophilized, and sealed to provide [4].

Manufacture of Products [5], [6], [7] and [8]

Excipient Sub-Mix

| Ingredient | Wt. % |
|---|---|
| Trehalose dihydrate | 20.773 |
| PVP K17 | 1.152 |
| Poloxamer 188 | 0.12 |
| Disodium Phos | 0 |
| Monosodium Phos | 0 |
| Water | 77.955 |

4.5 mg/ml fill product

| Ingredient | Wt. % |
|---|---|
| ZGN-440 | 0.450 |
| Poloxamer 188 | 0.240 |
| Trehalose Dihydrate | 10.387 |
| Povidone K17 | 0.576 |
| Disodium Phos | 0.591 |
| Monosodium Phos | 0.014 |
| Water | 87.742 |

Blank fill product

| Ingredient | Wt. % |
|---|---|
| ZGN-440 | 0 |
| Poloxamer 188 | 0.240 |
| Trehalose Dihydrate | 10.387 |
| Povidone K17 | 0.576 |
| Disodium Phos | 0.591 |
| Monosodium Phos | 0.014 |
| Water | 88.192 |

Microfluidization was performed on 13.2 mg/ml of ZGN-440 in a microfluidization (MF) vehicle containing 0.36% Poloxamer 188 in 40 mM phosphate buffer at pH 8.3. The suspension was then diluted with additional portions of the MF vehicle to a concentration of 9.0 mg/ml of ZGN-440. The resulting suspension (½ by weight) was combined with an excipient sub-mix (½ by weight, see table above) to give a 4.5 mg/ml fill product. This material (0.50 ml) was added to a vial, lyophilized, and sealed to provide [5].

The 4.5 mg/ml fill product (⅔ by weight) was combined with a blank fill product (⅓ by weight, see table above) to give a 3.0 mg/ml fill product. This material (0.5 ml) was added to a vial, lyophilized, and sealed to provide [6].

The 4.5 mg/ml fill product (⅓ by weight) was combined with a blank fill product (⅔ by weight) to give a 1.5 mg/ml fill product. This material (0.5 ml) was added to a vial, lyophilized, and sealed to provide [7].

The 4.5 mg/ml fill product (⅙ by weight) was combined with a blank fill product (⅚ by weight) to give a 0.75 mg/ml fill product. This material (0.5 ml) was added to a vial, lyophilized, and sealed to provide [8].

Filling was carried out by hand volumetric pipette with the pipette volume set to deliver 0.50 g on an analytical scale. Product vials for filling were sequentially numbered for each prototype group to allow product tracking throughout the duration of the filling process. Each fill product was analyzed by HPLC for ZGN-440 to determine ZGN-440 in solution measuring pH, ZGN-440 concentration of fill product and ZGN-440 concentration of supernatant of fill product. The filled product is held 30 minutes at ambient in the vials before all vials are loaded on to a −40° C. lyophilizer shelf. Because it takes approximately 15 minutes to fill 100 product vials, actual vials will sit 30-45 minutes prior to being placed on the −40° C. shelf where they will be held for 4 to 20 hours, freeze-dried, and sealed.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

What is claimed is:

1. A suspension lyophile composition suitable for subcutaneous administration to a patients upon reconstitution, comprising:
a crystalline form (e.g., Form A) of the compound 6-O-(4-dimethylaminoethoxy)cinnamoyl fumagillol, free base;
one or more bulking agents; and
one or more dispersing agents.

2. The suspension lyophile composition of claim 1, wherein the one or more dispersing agents are independently selected from poloxamer, polysorbate, Span, lecithin, Solutol H15 and Cremophor EL.

3. The suspension lyophile composition of claim 1 or 2, wherein the one or more dispersing agents include a poloxamer.

4. The suspension lyophile composition of any one of claims 1-3 wherein the poloxamer is poloxamer 188.

5. The suspension lyophile composition of claim 1 or 2, wherein the one or more dispersing agents include a polysorbate.

6. The suspension lyophile composition of claim 5, wherein the polysorbate is polysorbate 80, polysorbate 40 or polysorbate 20.

7. The suspension lyophile composition of claim 5 or 6, wherein the polysorbate is polysorbate 80.

8. The suspension lyophile composition of claim 1 or 2, wherein the one or more dispersing agents include Span 85, Span 80, Span 40 or Span 20.

9. The suspension lyophile composition of claim 1 or 2, wherein the one or more dispersing agents include Solutol H15.

10. The suspension lyophile composition of claim 1 or 2, wherein the one or more dispersing agents include Cremophor EL.

11. The suspension lyophile composition of claim 1 or 2, wherein the one or more dispersing agents include lecithin.

12. The suspension lyophile composition of any one of claims 1-11, wherein the one or more bulking agents is a sugar or derivative thereof.

13. The suspension lyophile composition of claim 12, wherein the one or more bulking agents are each independently selected from trehalose or hydrate thereof, mannitol, sucrose and raffinose.

14. The suspension lyophile composition of claim 12 or 13, wherein the one or more bulking agents include trehalose dihydrate.

15. The suspension lyophile composition of claim 12 or 13, wherein the one or more bulking agents include mannitol.

16. The suspension lyophile composition of claim 12 or 13, wherein the one or more bulking agents include sucrose.

17. The suspension lyophile composition of claim 12 or 13, wherein the one or more bulking agents include raffinose.

18. The suspension lyophile composition of any one of claims 1-17 wherein the crystalline form (e.g., Form A) has a particle size distribution profile suitable for use with a 27, 29 or 31-gauge needle.

19. The suspension lyophile composition of any one of claims 1-18, wherein the crystalline form (e.g., Form A) of the compound has a particle size distribution profile suitable for use with a 27, 29 or 31-gauge needle.

20. The suspension lyophile composition of any one of claims 1-19, wherein the composition comprises substantially minimal amorphous form of 6-O-(4-dimethylaminoethoxy)cinnamoyl fumagillol, free base.

21. The suspension lyophile composition of any one of claims 1-20, wherein the crystalline form (e.g., Form A) of the compound has a cumulative size distribution at 90% of less than about 40 μm upon reconstitution of the lyophile composition.

22. The suspension lyophile composition of any one of claims 1-21, further comprising one or more suspending agents.

23. The suspension lyophile composition of claim 22, wherein the one or more suspending agents are independently selected from polyvinylpyrrolidone, polyethylene glycol, sodium carboxymethyl cellulose and methylcellulose.

24. The suspension lyophile composition of claim 22 or 23, wherein the one or more suspending agents are independently selected from povidone K32, povidone K29, povidone K18, povidone K17 or povidone K12.

25. The suspension lyophile composition of any one of claims 22-24, wherein the one or more suspending agents include povidone K17.

26. The suspension lyophile composition of claim 22 or 23, wherein the one or more suspending agents include polyethylene glycol.

27. The suspension lyophile composition of claim 26, wherein the polyethylene glycol is PEG 3350 or PEG 4000.

28. The suspension lyophile composition of claim 22 or 23, wherein the one or more suspending agents include sodium carboxymethyl cellulose.

29. The suspension lyophile composition of claim 22 or 23, wherein the one or more suspending agents include methylcellulose.

30. The suspension lyophile composition of any one of claims 1-29, further comprising one or more buffer agents.

31. The suspension lyophile composition of claim 30, wherein the one or more buffer agents are each independently selected from monobasic sodium phosphate, dibasic sodium phosphate, monobasic potassium phosphate, dibasic potassium phosphate and hydrates thereof; and glutamic acid, glycine, arginine, meglumine, tromethamine, trolamine and salts thereof.

32. The suspension lyophile composition of claim 30 or 31, wherein the one or more buffer agents are each independently selected from monobasic sodium phosphate, monohydrate; dibasic sodium phosphate, heptahydrate; monobasic potassium phosphate, monohydrate and dibasic potassium phosphate dihydrate.

33. The suspension lyophile composition of any one of claims 1-32, wherein the pH of the composition is sufficient to minimize amount of the compound in solution.

34. The suspension lyophile composition of any one of claims 1-33, wherein the composition has a pH of about 6.5 to about 9 at 25° C.

35. The suspension lyophile composition of any one of claims 1-34, wherein the composition has a pH of about 7.8 to about 8.8 at 25° C.

36. The suspension lyophile composition of any one of claims 1-35, comprising about 70 to about 95 weight percent of the one or more bulking agents.

37. The suspension lyophile composition of any one of claims 1-36, comprising about 81 to about 88 weight percent of the one or more bulking agents.

38. The suspension lyophile composition of any one of claims 1-37, comprising about 2 to about 11 weight percent of the one or more dispersing agents.

39. The suspension lyophile composition of any one of claims 1-38, comprising about 0.6% to about 12% by weight of the crystalline form (e.g., Form A) of the compound.

40. A suspension lyophile composition suitable for subcutaneous administration to a patients upon reconstitution, comprising:
a crystalline form (e.g., Form A) of the compound 6-O-(4-dimethylaminoethoxy)cinnamoyl fumagillol, free base;
about 78 to about 88 weight percent trehalose dihydrate; and
about 2 to 3 weight percent poloxamer 188.

41. A suspension lyophile composition suitable for subcutaneous administration to a patients upon reconstitution, comprising:
a crystalline form (e.g., Form A) of the compound 6-O-(4-dimethylaminoethoxy)cinnamoyl fumagillol, free base;
about 81 to about 88 weight percent trehalose dihydrate;
about 2 to 3 weight percent poloxamer 188; and
optionally about 5 to 6 weight percent polysorbate 80, and wherein the suspension lyophile composition has a pH of about 7.8 to about 8.8 at 25° C.

42. A pre-loaded syringe comprising the suspension lyophile composition of any one of claims 1-41.

43. The pre-loaded syringe of claim 42, wherein the syringe is a dual chamber syringe and one chamber of the syringe contains the lyophile suspension composition.

44. A reconstitution vial comprising the suspension lyophile composition of any one of claims 1-41.

45. A ready to use reconstituted suspension composition comprising the suspension lyophile composition of any one of claims 1-41 and a reconstitution vehicle.

46. The ready to use reconstituted suspension composition of claim 45, wherein the reconstitution vehicle comprises water and non-ionic polymer.

47. The ready to use reconstituted suspension composition of claim 46, wherein the reconstitution vehicle comprises water and polysorbate 80.

48. The ready to use reconstituted suspension composition of claim 45, wherein the reconstitution vehicle comprises essentially water.

49. A unit dose vial or pre-loaded syringe for delivering about a 6 mg drug product dose, about a 4.5 mg drug product dose, about a 3.2 mg drug product dose, about a 2.8 mg drug product dose, about a 2.4 mg drug product dose, about a 1.8 mg drug product dose, about a 1.2 mg drug product dose, about a 0.6 mg drug product dose to a patient, or about a 0.3 mg drug product dose comprising the composition of any one of claims 1-48.

50. A reconstitution kit comprising:
a first container comprising a suspension lyophile composition comprising a crystalline form (e.g., Form A) of the compound 6-O-(4-dimethylaminoethoxy)cinnamoyl fumagillol, free base; and
a second container comprising a diluent for the suspension lyophile composition.

51. The reconstitution kit of claim 50, wherein upon reconstitution of the suspension lyophile composition with the diluent, the crystalline form (e.g., Form A) of the compound has a cumulative size distribution at 90% of less than about 200 μm.

52. The reconstitution kit of claim 50, wherein the crystalline form (e.g., Form A) of the compound has a cumulative size distribution at 90% of between about 15 μm to about 200 μm, or between about 15 μm to about 40 μm.

53. The reconstitution kit of any one of claims 50-52, wherein the diluent comprises water and polysorbate 80.

54. The reconstitution kit of any one of claims 50-52, wherein the diluent comprises essentially water.

55. The reconstitution kit of any one of claims 50-52, wherein the suspension lyophile composition further comprises trehalose dihydrate.

56. The reconstitution kit of any one of claims 50-52, wherein the suspension lyophile composition further comprises povidone K17.

57. The reconstitution kit of any one of claims 50-52, wherein the suspension lyophile composition further comprises poloxamer 188.

58. The reconstitution kit of any one of claims 50-52, wherein the suspension lyophile composition further comprises polysorbate 80.

59. A dual chamber cartridge, in which one of the chambers comprises the suspension lyophile composition of any one of claims 1-41 and the other chamber optionally comprises a diluent.
